(19) United States
(12) Patent Application Publication
(10) Pub. No.: US 2021/0403980 A1
(43) Pub. Date: Dec. 30, 2021

(54) METHODS OF MULTIPLEX dPCR ASSAYS AND SHORT-READ SEQUENCING ASSAYS

(71) Applicant: 13.8, Inc., Palo Alto, CA (US)

(72) Inventor: Christina FAN, San Jose, CA (US)

(21) Appl. No.: 17/206,692

(22) Filed: Mar. 19, 2021

Related U.S. Application Data
(63) Continuation of application No. PCT/US2019/052268, filed on Sep. 20, 2019.
(60) Provisional application No. 62/734,035, filed on Sep. 20, 2018.

(51) Int. Cl. C12Q 1/686; C12Q 1/6869

(52) U.S. Cl. CPC C12Q 1/686; C12Q 1/6869

(57) ABSTRACT

Provided herein are methods of multiplex digital PCR assays and short-read sequencing assays. The methods described herein utilize high-throughput sample distribution techniques, fluorescent channel monitoring, and nucleic acid sequence melting curve analysis.

### CROSS-REFERENCE

This application is a continuation of PCT/US2019/52268, filed Sep. 20, 2019, which claims priority to and the benefit of U.S. Provisional Application No. 62/734,035, filed Sep. 20, 2018, each of which is entirely incorporated herein by reference.

### SUMMARY

Provided herein are methods for digital PCR (dPCR) assays and short-read sequencing assays. The methods of dPCR assays as described herein comprise high-throughput, rapid distributing of a nucleic acid sample into millions or more partitions and monitoring of multiple fluorescent channels across individual partitions for a plurality of temperatures (e.g., during thermal ramping). The sequencing assays as described herein comprise aspects of these dPCR assays for determining short read sequences, and thus also comprise high-throughput, rapid distributing of a nucleic acid sample into millions or more partitions and monitoring of multiple fluorescent channels across individual partitions for a plurality of temperatures. Droplet-based, array-based, or polymerase colony-based dPCR platforms are described herein for use in conjunction with simultaneous fluorescence visualization individual partitions at a plurality of temperatures for high resolution melting temperature analysis. These methods can be used for, but are not limited to, mid-plex dPCR assays and high-plex dPCR assays to determine the presence or absence of a known target sequence in a sample or the quantification of the known target sequence in a sample, and short read sequencing to determine the sequence of a nucleic acid fragment from a sample. These methods can also be used for counting amplicons of a nucleic acid for quantitating a nucleic acid of interest in a sample, or quantitating multiple nucleic acids of interest in a sample. An instrument for three dimensional imaging of nucleic acids distributed into partitions (e.g., droplet-based, array based, or polymerase colony-based distribution) is also described. In some cases, this instrument comprises a device for controlling temperature and is used for monitoring melting of nucleic acids of interest from fluorescently labeled probes.

In some aspects, A method of nucleic acid sequence identification comprising: a) distributing a plurality of nucleic acid from a sample into a plurality of partitions, wherein a partition of the plurality comprises a single nucleic acid of the plurality, a polymerase, a plurality of primer sets, and a plurality of fluorescently labeled probes; b) amplifying the nucleic acids and amplicons thereof with the plurality of primer sets and the polymerase; c) hybridizing the fluorescently labeled probes to the nucleic acids or amplicons thereof, wherein a fluorescently labeled probe of the plurality comprises a region complementary to a region of a primer from the plurality of primer sets; and d) visualizing fluorescence of the fluorescently labeled probes in the partitions at a plurality of temperatures, wherein the fluorescence of the labeled probes at the plurality of temperatures of d) identifies the nucleic acid sequences of the partitions.

A method of nucleic acid sequence identification comprising: a) distributing a plurality of nucleic acid from a sample into a plurality of partitions such that at least 50% of the partitions comprise no more than one nucleic acid, and wherein the partitions further comprise a polymerase, a plurality of primer sets, and a plurality of fluorescently labeled probes; b) subjecting the partitions to amplification conditions to amplify the plurality of nucleic acids and produce double stranded amplicons using the plurality of primer sets and the polymerase; c) subjecting the partitions to amplification conditions so that only one primer of a primer set anneals to generate an excess of single stranded amplicons; d) incubating the single stranded amplicons at a temperature sufficient to anneal the plurality of fluorescently labeled probes to the single stranded amplicons; e) subjecting the annealed fluorescently labeled probes and single stranded amplicons to a plurality of temperatures; and f) visualizing fluorescence of the annealed fluorescently labeled probes while at the plurality of temperatures, wherein fluorescence at the plurality of temperatures of f) identifies the nucleic acid sequence of the nucleic acid of a partition. In some embodiments, a fluorescently labeled probe of the plurality of fluorescently labeled probes comprises a fluorophore and a quenching moiety. In some embodiments, the fluorophore is at the 5′ end of the probe and the quenching moiety is at the 3′ end of the probe or the fluorophore is at the 3′ end of the probe and the quenching moiety is at the 5′ end of the probe. In some embodiments, the fluorescently labeled probe of the plurality of fluorescently labeled probes comprises a hybridization probe pair, wherein a first probe of the hybridization probe pair comprises a 3′ fluorophore and a second probe of the hybridization pair probe comprises a 5′ fluorophore. In some embodiments, the plurality of partitions comprises at least 10⁵ partitions. In some embodiments, a fluorescently labeled probe of the plurality comprises a region complementary to a region of a primer from the plurality of primer sets. In some embodiments, the complementary region of a primer from at least one primer set of the plurality is not fully complementary with the region of the fluorescently labeled probe. In some embodiments, the complementary region of a primer from one primer set of the plurality is fully complementary with the region of the fluorescently labeled probe. In some embodiments, the complementary region of the primer is a unique identifier sequence. In some embodiments, each primer set of the plurality binds to a unique target nucleic acid sequence of a nucleic acid. In some embodiments, each primer set of the plurality comprises a target specific forward primer and a target specific reverse primer for a unique target nucleic acid sequence. In some embodiments, a target specific forward primer further comprises a universal sequence and an identifier sequence. In some embodiments, a target specific reverse primer further comprises a universal sequence and an identifier sequence. In some embodiments, an identifier sequence is unique for the target specific forward primer of a single primer set of the plurality. In some embodiments, an identifier sequence is unique for the target specific reverse primer of a single primer set of the plurality. In some embodiments, the unique identifier sequence comprises at least one nucleotide mismatch compared to a different unique identifier sequence. In some embodiments, the unique identifier sequence comprises a different nucleotide base content compared to a different unique identifier sequence. In some embodiments, the unique identifier sequence is different in nucleotide length compared to a different unique identifier sequence. In some embodiments, the unique identifier sequence has a different melting temperature when annealed to a fluorescently labeled probe than the different unique identifier sequence. In some embodiments, the plurality of temperatures comprise any temperature from 20° C. to 90° C. In some embodiments, the plurality of temperatures starts at 20° C. and is increased to 90° C. In some embodiments, each probe of plurality of fluorescently labeled probes is labeled with one fluorescent marker. In some embodiments, the plurality of fluorescently labeled probes comprises probes with different fluorescent markers. In some embodiments, the plurality of fluorescently labeled probes comprises a first probe with a first fluorescent marker, a second probe with a second fluorescent marker, a third probe with a third fluorescent marker, a fourth probe with a fourth fluorescent marker, or any combination thereof. In some embodiments, before step a), the nucleic acids are amplified. In some embodiments, the target specific forward primer is present in excess compared to the target specific reverse primer in step a). In some embodiments, the method is a high-throughput method. In some embodiments, the distributing is droplet-based, array-based, polymerase colony-based, or microfluidic device-based distribution. In some embodiments, the microfluidic device-base distribution uses microfluidic valves. In some embodiments, the visualizing fluorescence of the fluorescently labeled probes is simultaneous across the partitions at a temperature of the plurality of temperatures. In some embodiments, a target of a primer set of the plurality is a sequence of a cancer mutation. In some embodiments, the method further comprising quantitating a nucleic acid encoding a target sequence of the sample according to a number of partitions identified as comprising the nucleic acid of the sample. In some embodiments, the plurality of nucleic acids are DNA or RNA. In some embodiments, the more than 10 or 100 unique nucleic acid sequences are identified from the plurality of partitions. In some embodiments, all steps are performed in a single tube. In some embodiments, the partitions are configured in a 2D array. In some embodiments, the partitions are configured in a 3D volume. In some embodiments, the partitions are non-linearly configured. In some embodiments, the visualizing fluorescence of the fluorescently labeled probes is simultaneous across the plurality of partitions. In some embodiments, the visualizing fluorescence of the fluorescently labeled probes is non-linear. In some embodiments, the partitions are separated by physical partitions. In some embodiments, the partitions are separated by limited diffusion in a gel matrix. In some embodiments, the partitions are separated due to anchored primers and probes in a gel matrix or on beads.

In some aspects, a method of short-read sequencing comprises: a) attaching molecules having a common adaptor to a plurality of nucleic acid fragments from a sample to generate a plurality of adaptor-tagged nucleic acid fragments; b) distributing the plurality of adaptor-tagged nucleic acid fragments into at least 10⁵ partitions, wherein a partition of the 10⁵ partitions comprises a single adaptor-tagged nucleic acid fragment of the plurality, a first DNA polymerase, a second DNA polymerase, a primer set, a plurality of dNTPs, and a plurality of fluorescently labeled ddNTPs; c) amplifying the plurality of adaptor-tagged nucleic acids and amplicons thereof with the primer set and the first DNA polymerase; d) amplifying the plurality of adaptor-tagged nucleic acids and amplicons thereof with the primer set and the second DNA polymerase; and e) visualizing fluorescence of the fluorescently labeled ddNTPs in the partitions at a plurality of temperatures, wherein the fluorescence at the plurality of temperatures of e) identifies nucleotide bases and positions of the nucleotide bases in sequences of the nucleic acid fragments in the partitions for determining sequences of the nucleic acid fragments.

In some aspects, a method of short-read sequencing comprises: a) contacting a plurality of nucleic acid fragments from a sample to a plurality of molecules having a common adaptor to generate a plurality of adaptor-tagged nucleic acid fragments; b) distributing the plurality of adaptor-tagged nucleic acid fragments into at least 10⁵ partitions such that at least 50% of the at least 10⁵ partitions comprise no more than one adaptor-tagged nucleic acid fragment, and wherein the partitions further comprise a first DNA polymerase, a second DNA polymerase, a primer set, a plurality of dNTPs, and a plurality of fluorescently labeled ddNTPs; c) subjecting the partitions to amplification conditions that activate the first polymerase but not the second polymerase to amplify the plurality of adaptor-tagged nucleic acid sequences and produce amplicons using the primer set, the plurality of dNTPs, and the first polymerase; d) subjecting the partitions to amplification conditions that activate the second polymerase to amplify the amplicons, wherein one primer of the primer set is in excess compared to the second primer of the primer set; and e) visualizing fluorescence of ddNTPs at a plurality of temperatures in the plurality of partitions, wherein fluorescence at the plurality of temperatures of e) identifies a nucleotide base and position of the nucleotide base in a sequence of a nucleic acid fragment in a partition for determining the sequence of the nucleic acid fragment. In some embodiments, the attaching or contacting of molecules having a common adaptor is by ligation, in vitro transposition, or multiplex PCR. In some embodiments, the amplifying of c) uses dNTPs. In some embodiments, the primer set comprises a forward primer complementary to a region of the molecules having a common adaptor and a reverse primer complementary to a region of the molecules having a common adaptor. In some embodiments, the forward primer comprises a quenching moiety. In some embodiments, the quenching moiety is internal or at the 5′ end of the forward primer. In some embodiments, the forward primer comprises a cleavable site. In some embodiments, the cleavable site is a photocleavable site. In some embodiments, the quenching moiety is 3′ of the cleavable site. In some embodiments, the amplifying of d) uses dNTPs and ddNTPs. In some embodiments, the ddNTPs comprise a plurality of adenine ddNTPs comprising a first fluorescent label, a plurality of thymine ddNTPs comprising a second fluorescent label, a plurality of cytosine ddNTP comprising a third fluorescent label, and a plurality of guanine ddNTPs comprising a fourth fluorescent label. In some embodiments, forward primer is present in excess compared to the reverse primer in step d). In some embodiments, the annealing and extension temperature of step d) is raised compared to step c). In some embodiments, the forward primer comprises cleavage site. In some embodiments, the cleavage site is cleaved by ultraviolet light after step d). In some embodiments, the amplification of the nucleic acid fragment of step d) generates terminated nucleic acid sequence amplicons of varying lengths. In some embodiments, the terminated nucleic acid sequence amplicons of varying lengths have different melting temperatures based on their lengths. In some embodiments, the plurality of temperatures comprise any temperature from 20° C. to 90° C. In some embodiments, the plurality of temperatures starts at 20° C. and is increased to 90° C. In some embodiments, the method is high-throughput. In some embodiments, the distributing is droplet-based, array-based, polymerase colony-based, or microfluidic device-based distribution. In some embodiments, the microfluidic device-base distribution uses microfluidic valves. In some embodiments, the visualizing fluorescence of the fluorescently labeled ddNTPs is simultaneous across partitions at a temperature of the plurality of temperatures. In some embodiments, the plurality of nucleic acid fragments from the sample are from 10 to 25 nucleotides in length. In some embodiments, before step a) the plurality of nucleic acid fragments from the sample are produced by random shearing or enzymatic shearing. In some embodiments, the plurality of nucleic acid fragments are DNA or RNA. In some embodiments, all steps are performed in a single tube. In some embodiments, the partitions are configured in a 2D array. In some embodiments, the partitions are configured in a 3D volume. In some embodiments, the partitions are non-linearly configured. In some embodiments, the visualizing fluorescence of the fluorescently labeled ddNTPs is simultaneous across the plurality of partitions. In some embodiments, the visualizing fluorescence of the fluorescently labeled ddNTPs is non-linear.

In some aspects, a kit comprises: a) a plurality of molecules having a common adaptor; b) a first DNA polymerase; c) a second DNA polymerase; d) a primer set comprising a forward primer and a reverse primer, wherein the forward primer comprises a cleavage site and a quenching moiety; e) a plurality of dNTPs; and f) a plurality of fluorescently labeled ddNTPs.

In some aspects, a method for detecting a plurality of nucleic acids comprises contacting the plurality of nucleic acids to a plurality of fluorescently labeled probes, wherein the plurality of fluorescently labeled probes hybridize to the plurality of nucleic acids; and detecting the fluorescently labeled probes using a three dimensional imaging device. In some embodiments, the method further comprises distributing the plurality of nucleic acids from a sample into a plurality of partitions such that at least 50% of the partitions comprise no more than one nucleic acid, and wherein the partitions further comprise a polymerase, a plurality of primer sets, and a plurality of fluorescently labeled probes before the contacting. In some embodiments, the method further comprises: subjecting the partitions to amplification conditions to amplify the plurality of nucleic acid sequences and produce double stranded amplicons using the plurality of primer sets and the polymerase; subjecting the partitions to amplification conditions so that only one primer of a primer set anneals to generate an excess of single stranded amplicons; incubating the single stranded amplicons at a temperature sufficient to anneal the plurality of fluorescently labeled probes to the single stranded amplicons; subjecting the annealed fluorescently labeled probes and single stranded amplicons to a plurality of temperatures; and visualizing fluorescence of the annealed fluorescently labeled probes while at the plurality of temperatures, wherein fluorescence at the plurality of temperatures of the visualizing step identifies a nucleic acid sequence of a partition.

In some aspects, a method of counting a number of nucleic acids encoding a first sequence in a sample comprising: a) distributing a plurality of nucleic acids from a sample into a plurality of partitions such that at least 50% of the partitions comprise no more than one nucleic acid, and wherein the partitions further comprise a polymerase, a primer set, and a plurality of fluorescently labeled probes; b) subjecting the partitions to amplification conditions to amplify the nucleic acid encoding the first sequence and produce double stranded amplicons using the primer set and the polymerase; c) subjecting the partitions to amplification conditions so that only one primer of a primer set anneals to generate an excess of single stranded amplicons; d) incubating the single stranded amplicons at a temperature sufficient to anneal the plurality of fluorescently labeled probes to the single stranded amplicons; e) visualizing the plurality of partitions for fluorescence of the annealed fluorescently labeled probes, and f) counting a number of fluorescent partitions, wherein a fluorescent partition identifies the presence of the nucleic acid encoding the first sequence.

In some aspects, a method of counting numbers of a first plurality of nucleic acids encoding a first plurality of sequences comprising: a) distributing a second plurality of nucleic acids from a sample into a plurality of partitions such that at least 50% of the partitions comprise no more than one nucleic acid of the second plurality of nucleic acids, and wherein the partitions further comprise a polymerase, a plurality of primer sets for the first plurality of nucleic acid molecules, and a plurality of fluorescently labeled probes; b) subjecting the partitions to amplification conditions to amplify the first plurality of nucleic acids and produce double stranded amplicons using the plurality of primer sets and the polymerase; c) subjecting the partitions to amplification conditions so that only one primer of a primer set anneals to generate an excess of single stranded amplicons; d) incubating the single stranded amplicons at a temperature sufficient to anneal the plurality of fluorescently labeled probes to the single stranded amplicons; e) subjecting the annealed fluorescently labeled probes and single stranded amplicons to a plurality of temperatures; and f) visualizing fluorescence of the annealed fluorescently labeled probes while at the plurality of temperatures, g) counting a number of fluorescent partitions, wherein a fluorescent partition identifies the presence of a nucleic acid from first plurality of nucleic acids encoding a first plurality of sequences.

In some aspects, a method of monitoring melting of single stranded amplicons from fluorescently labeled probes comprises: a) distributing a plurality of nucleic acids from a sample into a plurality of partitions such that at least 50% of the partitions comprise no more than one nucleic acid, and wherein the partitions further comprise a polymerase, a plurality of primer sets for the first plurality of nucleic acid molecules, and a plurality of fluorescently labeled probes; b) subjecting the partitions to amplification conditions to amplify the first plurality of nucleic acids and produce double stranded amplicons using the plurality of primer sets and the polymerase; c) subjecting the partitions to amplification conditions so that only one primer of a primer set anneals to generate an excess of the single stranded amplicons; d) incubating the single stranded amplicons at a temperature sufficient to anneal the plurality of fluorescently labeled probes to the single stranded amplicons; e) subjecting the annealed fluorescently labeled probes and single stranded amplicons to a plurality of temperatures; and f) monitoring the fluorescence of the annealed fluorescently labeled probes while at the plurality of temperatures, wherein a loss of fluorescence at a temperature of the plurality of temperatures in a partition indicates melting of a single stranded amplicon from a fluorescently labeled probe. In some embodiments, the visualizing or monitoring is by a three dimensional imaging device. In some embodiments, the three dimensional imaging device is a light sheet imaging device. In some embodiments, the plurality of partitions are in a tube. In some embodiments, the tube is in a chamber. In some embodiments, the three dimensional imaging device is connected to a device comprising the chamber, wherein the device controls the temperature of the tube. In some embodiments, the device heats the tube. In some embodiments, the device cools the tube.

In some aspects, a system comprises a three dimensional imaging device and a plurality of nucleic acid from a sample in a plurality of partitions, wherein a partition of the plurality comprises a single nucleic acid sequence of the plurality, a polymerase, a plurality of primer sets, and a plurality of fluorescently labeled probes. In some embodiments, the three dimensional imaging device is a light sheet imaging device. In some embodiments, the plurality of partitions are in a tube. In some embodiments, the system further comprises a chamber to hold the tube. In some embodiments, the system further comprises a device that controls the temperature of the tube. In some embodiments, the device heats the tube. In some embodiments, the device cools the tube.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the advantages of digital PCR (dPCR) assays and short-read sequencing assays described herein over commercial dPCR and next generation sequencing (NGS);

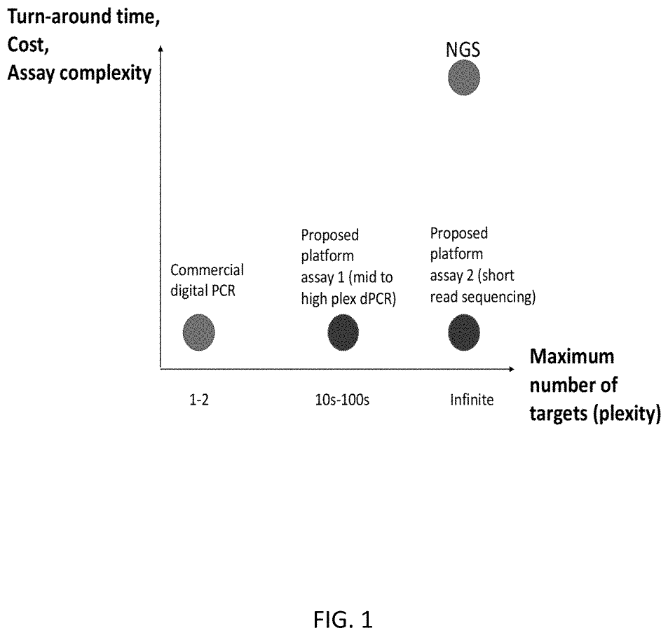

FIG. 2 shows a mid-plex dPCR assay work flow for three target sequences using a probe with a single fluorescent label;

FIG. 3 shows a high-plex dPCR assay work flow for 400 target sequences using four different fluorescent labels for the probes and identifier sequences with up to 10 sequence mismatches to a probe sequence (and thus 10 possible melting temperatures).

FIG. 4 shows short-read sequencing assay work flow for a nucleic acid fragment.

### DETAILED DESCRIPTION

The present disclosure provides methods of multiplex digital polymerase chain reaction (dPCR) assays and sequencing assays utilizing melting curve analysis. These methods are fast and simple, without the need to batch with other samples. Furthermore, these methods can allow for single molecule sensitivity and absolute quantitation. Additionally, these methods can utilize targeted panels or short sequence reads for identification of nucleic acid sequences within a sample.

The disclosure herein provides advantages over current PCR assays and sequencing assays. For example, real-time PCR assays are fast and simple, and current dPCR assays allow for single molecule sensitivity and absolute quantitation, but both real-time PCR assays and current dPCR assays are limited to a few targets per assay due to the availability of fluorescent channels. Although next-generation sequencing (NGS) assays can allow for examination of large number of targets simultaneously for various sequencing-based clinical assays, NGS assays can be complex, with long turn-over time, and can require multiple samples to be pooled together for a sequencing run to achieve desirable assay cost.

Therefore, multiplex digital polymerase chain reaction (dPCR) assays and sequencing assays as described herein can provide fast and simple assays that allow for large numbers of targets, single nucleic acid sequence sensitivity, and absolute quantitation that is also relatively cheap for use in routinely performed clinical assays. A dPCR assay as described herein can identify tens to hundreds of target sequences in a single tube. For example, the dPCR assay described herein is used to identify tens to hundreds of target sequences in targeted panels for cancer DNA mutation, targeted panels to detect epigenetic changes (e.g., dPCR after bisulfite conversion), or targeted panels for non-invasive prenatal diagnosis of aneuploidy. A sequencing assay as described herein can be a short-read sequencing assay in a single tube. This short read sequencing assay can be used for short read sequencing assays such as whole genome shot gun sequencing for non-invasive prenatal diagnosis of aneuploidy or RNA-seq applications for gene expression analysis of tissues. This short read sequencing assay can also be used for short read sequencing assays for molecular typing of viral and microbial strains, for cancer mutation analysis, and for copy number variation (CNV) analysis.

dPCR Assays and Sequencing Assays

The methods of dPCR assays as described herein can comprise high-throughput, rapid distributing of nucleic acid sequences of a sample into millions or more partitions and monitoring of multiple fluorescent channels for individual partitions for a plurality of temperatures (e.g., during thermal ramping). The sequencing assays as described herein can comprise aspects of these dPCR assays for determining short read sequences, and thus also can comprise high-throughput, rapid distributing of a nucleic acid sequences of a sample into millions or more partitions and monitoring of multiple fluorescent channels for individual partitions while the temperature is changed. Most types of digital PCR platforms can be used with the dPCR assays described herein: droplet-based, array-based (microwell array, patterned planar or 3D array, droplet array), bead-based, polymerase colony-based in 2D or 3D gel substrates. After distribution to partitions occurs, there can be no fluid exchange with the partitions (e.g., no merging, splitting, or breaking of droplets) and there can be no user intervention. Combined, these can enable high resolution melt curve analysis for an individual partition after digital amplification of single nucleic acid sequences at a rate of more than 20,000, 30,000, 40,000, 50,000 or 10⁵, 10⁶, or more nucleic acid sequences simultaneously. These methods can be used for, but are not limited to, mid-plex dPCR assays, high-plex dPCR assays, and short read sequencing, in which there can be a fast turn-around time for completion as these assays can be mainly driven by a single PCR reaction.

Notably, none of the currently available digital PCR platforms can achieve the needed throughput of distribution to partitions and multiple fluorescent channel monitoring during thermal ramping. The current highest achievable number is about 20,000. Additionally, none of the current droplet-based platforms (e.g., BioRad, RainDance) can offer real-time fluorescence monitoring, thus melt-curve analysis is not possible. For array-based platforms (e.g., Fluidigm's Biomark), the number of partitions can be limited to upper hundreds. Additionally, array-based platforms (e.g., Thermo's QuantStudio 3D) decouple thermal cycling from fluorescence reading, thus can also not perform melt-curve analysis of a plurality of partitions due to its design. Furthermore, commercial dPCR platforms only can have 2 available fluorescent channels, limiting assays to 2-plex.

As described herein, a method of nucleic acid sequence identification can comprise: a) distributing a plurality of nucleic acids from a sample into a plurality of partitions, wherein a partition of the plurality comprises a single nucleic acid of the plurality, a polymerase, a plurality of primer sets, and a plurality of fluorescently labeled probes; b) amplifying the nucleic acids and amplicons thereof with the plurality of primer sets and the polymerase; c) hybridizing the fluorescently labeled probes to the nucleic acids or amplicons thereof, wherein a fluorescently labeled probe of the plurality comprises a region complementary to a region of a primer from the plurality of primer sets; and c) visualizing fluorescence of the fluorescently labeled probes in the partitions at a plurality of temperatures; wherein the fluorescence of the labeled probes at the plurality of temperatures of d) identifies the nucleic acid sequences of the nucleic acids of the partitions. Alternatively, a method of nucleic acid sequence identification comprises: a) distributing a plurality of nucleic acids from a sample into a plurality of partitions such that at least 50% of the partitions comprise no more than one nucleic acid, and wherein the partitions further comprise a polymerase, a plurality of primer sets, and a plurality of fluorescently labeled probes; b) subjecting the partitions to amplification conditions to amplify the plurality of nucleic acids and produce double stranded amplicons using the plurality of primer sets and the polymerase; c) subjecting the partitions to amplification conditions so that only one primer of a primer set anneals to generate an excess of single stranded amplicons; d) incubating the single stranded amplicons at a temperature sufficient to anneal the plurality of fluorescently labeled probes to the single stranded amplicons; e) subjecting the annealed fluorescently labeled probes and single stranded amplicons to a plurality of temperatures; and f) visualizing fluorescence of the annealed fluorescently labeled probes while at the plurality of temperatures; wherein fluorescence at the plurality of temperatures of g) identifies a nucleic acid sequence of a nucleic acid of a partition.

As described herein, a method of short-read sequencing comprises: a) attaching molecules having a common adaptor to a plurality of nucleic acid fragments from a sample to generate a plurality of adaptor-tagged nucleic acid fragments; b) distributing the plurality of adaptor-tagged nucleic acid fragments into at least 10⁵ partitions, wherein a partition of the 10⁵ partitions comprises a single adaptor-tagged nucleic acid fragment of the plurality, a first DNA polymerase, a second DNA polymerase, a primer set, a plurality of dNTPs, and a plurality of fluorescently labeled ddNTPs; c) amplifying the plurality of adaptor-tagged nucleic acid sequences and amplicons thereof with the primer set and the first DNA polymerase; d) amplifying the plurality of adaptor-tagged nucleic acid fragments and amplicons thereof with the primer set and the second DNA polymerase; and e) visualizing fluorescence of the fluorescently labeled ddNTPs in the partitions at a plurality of temperatures; wherein the fluorescence at the plurality of temperatures of e) identifies nucleotide bases and positions of the nucleotide bases in sequences of the nucleic acid fragments in the partitions for determining sequences of the nucleic acid fragments. Alternatively, a method of short-read sequencing comprises: a) contacting a plurality of nucleic acid fragments from a sample to a plurality of molecules having a common adaptor to generate a plurality of adaptor-tagged nucleic acid fragments; b) distributing the plurality of adaptor-tagged nucleic acid fragments into at least 10⁵ partitions such that at least 50% of the at least 10⁵ partitions comprise no more than one adaptor-tagged nucleic acid sequence, and wherein the partitions further comprise a first DNA polymerase, a second DNA polymerase, a primer set, a plurality of dNTPs, and a plurality of fluorescently labeled ddNTPs; c) subjecting the partitions to amplification conditions that activate the first polymerase but not the second polymerase to amplify the plurality of adaptor-tagged nucleic acid sequences and produce amplicons using the primer set, the plurality of dNTPs, and the first polymerase; d) subjecting the partitions to amplification conditions that activate the second polymerase to amplify the amplicons, wherein one primer of the primer set is in excess compared to the second primer of the primer set; and e) visualizing fluorescence of ddNTPs at a plurality of temperatures in the plurality of partitions; wherein fluorescence at the plurality of temperatures of e) identifies a nucleotide base and position of the nucleotide base in a sequence of a nucleic acid fragment in a partition for determining the sequence of the nucleic acid fragment. A method for detecting a plurality of nucleic acids comprises contacting the plurality of nucleic acids to a plurality of fluorescently labeled probes, wherein the plurality of fluorescently labeled probes hybridize to the plurality of nucleic acids; and detecting the fluorescently labeled probes using a three dimensional imaging device. In some embodiments, the method of detecting a plurality of nucleic acids further comprises distributing the plurality of nucleic acids from a sample into a plurality of partitions such that at least 50% of the partitions comprise no more than one nucleic acid, and wherein the partitions further comprise a polymerase, a plurality of primer sets, and a plurality of fluorescently labeled probes before the contacting. In some embodiments, the method of detecting a plurality of nucleic acids further comprises: subjecting the partitions to amplification conditions to amplify the plurality of nucleic acid sequences and produce double stranded amplicons using the plurality of primer sets and the polymerase; subjecting the partitions to amplification conditions so that only one primer of a primer set anneals to generate an excess of single stranded amplicons; incubating the single stranded amplicons at a temperature sufficient to anneal the plurality of fluorescently labeled probes to the single stranded amplicons; subjecting the annealed fluorescently labeled probes and single stranded amplicons to a plurality of temperatures; and visualizing fluorescence of the annealed fluorescently labeled probes while at the plurality of temperatures, wherein fluorescence at the plurality of temperatures of the visualizing step identifies a nucleic acid sequence of a partition. Methods of counting or quantitating the number of nucleic acids of interest from a sample are also described herein. For example, a method of counting a number of nucleic acids encoding a sequence of interest in a sample comprises: distributing a plurality of nucleic acids from a sample into a plurality of partitions such that at least 50% of the partitions comprise no more than one nucleic acid, and wherein the partitions further comprise a polymerase, a primer set, and a plurality of fluorescently labeled probes; subjecting the partitions to amplification conditions to amplify the nucleic acid encoding the first sequence and produce double stranded amplicons using the primer set and the polymerase; subjecting the partitions to amplification conditions so that only one primer of a primer set anneals to generate an excess of single stranded amplicons; incubating the single stranded amplicons at a temperature sufficient to anneal the plurality of fluorescently labeled probes to the single stranded amplicons; visualizing the plurality of partitions for fluorescence of the annealed fluorescently labeled probes, and counting a number of fluorescent partitions, wherein a fluorescent partition identifies the presence of the nucleic acid encoding the first sequence. Alternatively, a method of counting numbers of a first plurality of nucleic acids encoding a plurality of sequences of interest comprises: distributing a second plurality of nucleic acids from a sample into a plurality of partitions such that at least 50% of the partitions comprise no more than one nucleic acid of the second plurality of nucleic acids, and wherein the partitions further comprise a polymerase, a plurality of primer sets for the first plurality of nucleic acid molecules, and a plurality of fluorescently labeled probes; subjecting the partitions to amplification conditions to amplify the first plurality of nucleic acids and produce double stranded amplicons using the plurality of primer sets and the polymerase; subjecting the partitions to amplification conditions so that only one primer of a primer set anneals to generate an excess of single stranded amplicons; incubating the single stranded amplicons at a temperature sufficient to anneal the plurality of fluorescently labeled probes to the single stranded amplicons; subjecting the annealed fluorescently labeled probes and single stranded amplicons to a plurality of temperatures; and visualizing fluorescence of the annealed fluorescently labeled probes while at the plurality of temperatures, counting a number of fluorescent partitions, wherein a fluorescent partition identifies the presence of a nucleic acid from first plurality of nucleic acids encoding a first plurality of sequences. Also disclosed herein are methods of monitoring melting of nucleic acids of interest or amplicons thereof from a fluorescent probe. For example, a method of monitoring melting of single stranded amplicons (e.g., amplicon of a nucleic acid of interest) from fluorescently labeled probes comprises: distributing a plurality of nucleic acids from a sample into a plurality of partitions such that at least 50% of the partitions comprise no more than one nucleic acid, and wherein the partitions further comprise a polymerase, a plurality of primer sets for the first plurality of nucleic acid molecules, and a plurality of fluorescently labeled probes; subjecting the partitions to amplification conditions to amplify the first plurality of nucleic acids and produce double stranded amplicons using the plurality of primer sets and the polymerase; subjecting the partitions to amplification conditions so that only one primer of a primer set anneals to generate an excess of the single stranded amplicons; incubating the single stranded amplicons at a temperature sufficient to anneal the plurality of fluorescently labeled probes to the single stranded amplicons; subjecting the annealed fluorescently labeled probes and single stranded amplicons to a plurality of temperatures; and monitoring the fluorescence of the annealed fluorescently labeled probes while at the plurality of temperatures, wherein a loss of fluorescence at a temperature of the plurality of temperatures in a partition indicates melting of a single stranded amplicon from a fluorescently labeled probe. Furthermore, described herein is a system for carrying out the methods described herein. For example, a system comprises a three dimensional imaging device and a plurality of nucleic acid from a sample in a plurality of partitions, wherein a partition of the plurality comprises a single nucleic acid sequence of the plurality, a polymerase, a plurality of primer sets, and a plurality of fluorescently labeled probes. Often, the three dimensional imaging device is a light sheet imaging device. In some embodiments, the plurality of partitions are in a tube. Sometimes, the system further comprises a chamber to hold the tube. Sometimes, the system further comprises a device that controls the temperature of the tube. Often, the device heats the tube. Often, the device cools the tube.

High-throughput, rapid distribution of a plurality of nucleic acid sequences into a plurality of partitions. Several methods can be used for the high-throughput, rapid distribution of a plurality of nucleic acid sequences from a sample into a plurality of partitions, wherein a partition of the plurality comprises a single nucleic acid sequence of the plurality. For example, high-throughput, rapid distribution of nucleic acid sequences from a sample into a plurality of partitions can be generated by high-throughput droplet generation, by spreading of a nucleic acid sample across a microcapillary array plate, or by separation into partitions in a microfluidic device (e.g., by microfluidic valves in the microfluidic device), by spreading nucleic acid across a patterned planar surface or patterned 3D surface (e.g. surface of spheres or volumes inside porous beads), or in a hydrogel or polymer solution later subject to gellation. A partition can be physical partition or a partition generated by limited diffusion. In gels, for example, there are no physical partitions, but the amplification of individual molecule are instead partitioned within the polymer by limited diffusion. In some embodiments, one or more primers or probes have an anchor moiety that covalently links the primers and probes to the polymer matrix.

High-throughput droplet generation can be performed using various techniques. One technique is performed by generating millions of droplets via microfluidic devices and utilizing hydrodynamic focusing, whereby oil and aqueous phases meet at a T junction. Such devices can be fabricated using soft lithography. Starting volume, flow rate, channel size, time of droplet generation etc., can be used to control the number and size of the droplets. Alternatively, a microcapillary plate can be fitted on a microcentrifuge tube to generate 285,000 65-micron diameter droplets from a volume of 16-20 ul in <6 minutes via centrifugation (Chen, Zitian, et al. Lab on a Chip 17.2 (2017): 235-240). The number of partitions can be 10-fold more than any current commercial systems. A benchtop centrifuge with speed of >7000 g can be used. By increasing total sample volume and reducing droplet size by using higher centrifugation speed and smaller capillary, 2.8 million to 5.6 million droplets or partitions can be generated, enabling examination of 10⁵ to 10⁶ single nucleic acid sequences from a single sample. This range of nucleic acid sequences can allow for sufficient read depth to identify a nucleic acid sequence from a single sample in a targeted sequencing or shallow whole shotgun sequencing assay.

Spreading of a sample of nucleic acid sequences across a microcapillary array plate can be performed using various techniques. One such technique can utilize microcapillary array plates designed for optical applications. For example, microcapillary array plates that use glass drawing technology produce hollow arrays in a range of cross sections, pore sizes, and lengths. These plates can have millions of openings and be made of glass (e.g., hydrophilic). The solution comprising nucleic acid sequences can be spread across the plate and the solution can passively fill in to the openings. Surface tension can hold the solution in an individual opening or partition in place and prevents cross-talk across openings or partitions.

Separation of a sample of nucleic acid sequences into partitions of a microfluidic device can be performed using various techniques. One such technique can utilize the intersection of control channels and flow channels when the control channels are pressurized to create microfluidic valves for partitioning nucleic acid sequences in a sample. This can create complete seals as result of the thin membranes separating the two channels being deflected into the flow channels. Such microfluidic devices can be fabricated using soft lithography in which a soft polymer, such as polydimethylsiloxane (PDMS), is cast onto a mold that contains a microfabricated relief or engraved pattern comprising microfluidic valves.

The partitions can be non-linearly configured. The partitions can be configured in a 2D array. A 2D array can be planar. A 2D array can be configured on a slide. The partitions can be configured in a 3D volume. A 3D volume can be in a flow cell. A 3D volume can be in a tube.

Monitoring fluorescent channels during thermal ramping. Several methods of monitoring of one or more fluorescent channels for a plurality of partitions generated by the high-throughput, rapid distribution as described above at a plurality of temperatures or during thermal ramping can be used. For example, monitoring of one or more fluorescent channels for individual partitions can be performed using 3D imaging of emulsions or gel substrates or bead suspensions, planar imaging of emulsions or gel substrates or bead suspensions, or planar imaging of an array plate or patterned substrate comprising the partitioned nucleic acid sequences.

3D imaging of emulsions or substrate volume (e.g. gel) or bead suspensions for visualizing multiple fluorescent channels for individual partitions at a plurality of temperatures or during thermal ramping can be performed using various techniques. For example, a compact scanning confocal fluorescence microscope in a system that can mechanically move a cuvette comprising a sample can be used for the detection of particles as they traverse the confocal volume, utilizing pattern recognition of spikes in the emission intensity of data for sample analysis, can be used as a 3D fluorescent particle detector. Another example of 3D imaging is lightsheet imaging. The sample traverses across the light sheet, and fluorescence of each cross-section across the tube is recorded. Another 3D imaging techniques include 2-photon microscopy and photoactivated localization microscopy. These techniques can be applied to partitions comprising nucleic acid sequences, and can be modified so that the temperature of the sample in the tube can be controlled (e.g. thermal ramping) during recording of fluorescence of the individual partitions for digital melt curve analysis. Temperature control can be done by contacting the 3D substrate or sample tube with a metal holder controlled by a Peltier element, or by placing the sample tube in a liquid solution heated by a Peltier element.

Planar imaging of emulsions or an array plate for visualizing one or more fluorescent channels for across a plurality of partitions at a plurality of temperatures or during thermal ramping can be performed using various techniques. For distribution into partitions via droplets, emulsion can be spread out on a slide or flow cell such that a single layer of droplet is formed. The slide can then be heated on a flat top Peltier block (e.g., thermal ramping), and the fluorescence of droplets can be monitored by imaging the slide. For distribution to partitions via a microcapillary array plate, the plate can be heated on a flat top Peltier block (e.g., thermal ramping), and the fluorescence of partitions can be monitored by imaging of the plate. For distribution to partitions in gel substrates, or patterned 2D surfaces, or microwells, the plate can be heated on a flat top Peltier block (e.g., thermal ramping), and the fluorescence of partitions can be monitored by imaging of the plate.

Visualizing of the fluorescence across a plurality of partitions at a plurality of temperatures or during thermal ramping can be nonlinear detection. Visualizing of the fluorescence across a plurality of partitions at a plurality of temperatures or during thermal ramping can be nonordered detection. Visualizing of the fluorescence cross a plurality of partitions at a plurality of temperatures or during thermal ramping can be nonlinear and nonordered detection. The detection can be simultaneous across a plurality of partitions at a temperature of the plurality of temperatures. The partitions can be visualized without being positioned in a line that is then flowed through a detector (e.g., without being flowed through the detector one partition at a time).

Single Nucleic Acid Sequence Melt Curve Analysis. Single nucleic acid sequence melt curve analysis coupled with monitoring of one or more fluorescent channels can be used to identify a target sequence or identify the nucleic acid sequence of a sample. For example, one can use melting temperature (Tm) of the PCR product or probe/target hybrid as an additional dimension to code for the target sequence. For multi-plex PCR, the number of possible target sequences in a single tube assay can be the number of fluorescent channels multiplied by the number of different Tm. For example, four fluorescent channels with a probe for a fluorescent channel comprising 10 Tm, a total of 40 different target sequences can be assayed. As another example, two different probes (the first probe can be labeled with a first fluorescent label of two different fluorescent labels and comprises 10 Tm, and the second probe can be labeled with a second fluorescent label of the two different fluorescent labels, in which the first fluorescent label and the second fluorescent label are different fluorescent labels, and comprises 10 Tm) a total of 400 different target sequences can be assayed. For short-read sequencing, the Tm peak associated with a molecule terminated by ddNTP of a fluorescent label in relation to the Tm peaks of other molecules terminated by ddNTPs of fluorescent labels can be used to identify the nucleic acid base (e.g., adenine, thymine, cytosine, or guanine) and its position in the nucleic acid fragment of that individual partition.

Mid-Plex dPCR Assay Workflow

A dPCR assay as described above can be a mid-plex dPCR assay. A mid-plex dPCR assay can be performed with tens of target sequences. For example, a mid-plex dPCR assay can be performed with 10, 20, 30, 40, 50, 60, 70, 80, 90, or more target sequences. As one example, a nucleic acid sample comprising a plurality of nucleic acid sequences can be pooled together into a single tube. DNA polymerase and target specific primers for each target of interest can be added to the tube, and the plurality of nucleic acid sequences can be extended. After the extension, excess primers can be removed.

Subsequently, a universal primer comprising a universal sequence, an identifier sequence, a target specific forward primer, and a target specific reverse primer can be added to the tube for each target of interest. The identifier sequence of the universal primer is different for each target sequence. There can be an excess of the universal primers compared to the target specific reverse primers. Additionally, a plurality of probes with a fluorescent label can be added to the tube. A probe of the plurality of probes can comprise a fluorescent label on one end of the probe and a fluorescence quenching moiety on the other end of the probe. For example, a probe has a fluorescent label on the 5′ end of the probe and a quenching moiety on the 3′ end of the probe. Alternatively, a probe can have a quenching moiety on the 5′ end of the probe and a fluorescent label on the 3′ end of the probe. The quenching moiety can quench the fluorescence of the fluorescent label when the probe is in single-stranded form (e.g., not hybridized to a complementary nucleotide sequence), but does not quench the fluorescence of the fluorescent label with the probe is hybridized to a complementary nucleotide strand. The plurality of probes, in some cases, comprises a plurality of a hybridization probe pair. A hybridization probe pair can comprise a first probe comprising a fluorophore on the 3′ end and a second probe comprising a fluorophore on the 5′ end. A fluorescence can be generated by exciting the 3′ fluorophore on the first probe and then detecting fluorescence from the 5′ fluorophore on the second probe (e.g., via Forster resonance energy transfer (FRET)). A hybridization probe pair can hybridize to a complementary nucleotide strand wherein the 3′ fluorophore is next to the 5′ fluorophore when the hybridization probe pair hybridizes to the complementary nucleotide strand. A probe can also comprise a modified base or locked nucleic acid (LNA) to enhance the difference in melting temperature between different identifier sequences. Importantly, for example, a probe labeled with a first fluorescent label can comprise a probe sequence that is completely complementary to an identifier sequence of a first universal primer, complementary except for one mismatch to an identifier sequence of a second universal primer, complementary except for two mismatches to an identifier sequence of a third universal primer, etc. Alternatively, a probe labeled with a first fluorescent label can comprise a probe sequence that is completely complementary to an identifier sequence of a first universal primer resulting in a first melting temperature, complementary to an identifier sequence of a second universal primer resulting in a second melting temperature, complementary to an identifier sequence of a third universal primer resulting in a third melting temperature, etc. (this can be achieved by having identifier sequence and probe sequence of different lengths or sequence compositions (e.g., nucleotide base content)). In some embodiments, the first melting temperature, the second melting temperature, the third melting temperature, etc. are different melting temperatures. The sample in the tube then can undergo digital distribution to partitions as described above (e.g., either by high-throughput droplet generation or by spreading of nucleic acid sequences of the sample across a microcapillary array plate) to distribute the nucleic acid sequences into an individual partition (e.g., a partition of a plurality of partitions comprises a single nucleic acid sequence). Next, nucleic acid sequences in individual partitions undergo a symmetric phase PCR amplification to generate double stranded products with the universal primer and the target specific reverse primer. Then, the individual partitions can undergo an asymmetric phase of PCR amplification when the target specific reverse primer has been completely used up to generate a single stranded product. The probes can then hybridize to the identifier sequence of the single stranded amplified target sequence, and the partitions can then fluoresce if the probe is hybridized. Absence of fluorescence can indicate that no amplified target sequence is found in that individual partition.

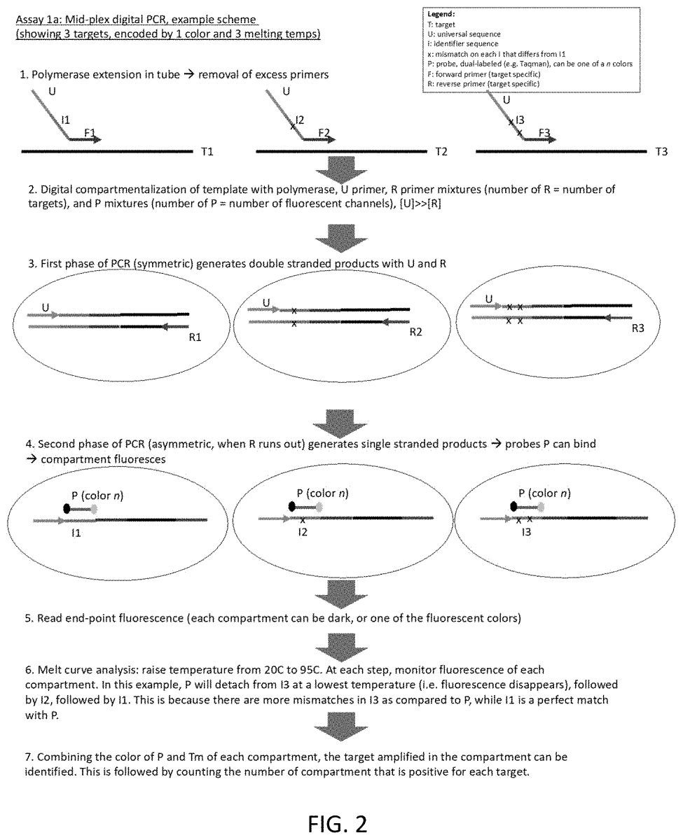

Next, fluorescence can be visualized simultaneously across the plurality of partitions at a plurality of temperatures or during thermal ramping. Thermal ramping can comprise increasing the temperature at intervals of 0.1, 0.2, 0.3, 0.4, 0.5, 0.6, 0.7, 0.8, 0.9, or 1° C. In some examples, the interval is 1, 2, 3, 4, 5, 6, 7, 8, 9, or 10 seconds. For example, a plurality of temperatures can be any temperature from 10° C. to 105° C., 15° C. to 100° C., or 20° C. to 95° C. A plurality of temperatures can be temperatures from thermal ramping, such as increasing a temperature from 10° C. to 105° C., 15° C. to 100° C., or 20° C. to 95° C. At different temperatures, the probes can detach from the identifier sequence of the amplified target sequence with the probes comprising the lowest complementarity detaching at the lowest temperatures, causing the fluorescence to disappear. The probes can detach from the identifier sequence of the amplified target sequence with the next lowest complementarity at a higher temperature, and so on, causing the fluorescence to disappear when the probes detach. The disappearance of fluorescence can be the result of the probe being in single-stranded form, in which the distance between the fluorescent label and the quencher moiety is closer due to the coiling of the probe. Therefore, by combining the color of the fluorescence/loss of fluorescence and the melting temperature of an individual partition, the target amplified sequence in an individual partition can be identified. This can then be followed by counting or quantitating the number of partitions that were positive for a specific identified amplified target sequence. FIG. 2 illustrates this work flow for three target sequences using a probe with a single fluorescent label. In some examples, thermal ramping and fluorescence detection occurs in less than 4, 6, 8, 10, 12, 14, 16, 20, 24, 28, 32, 36, 40, 44, or 48 hours.

High-Plex dPCR Assay Workflow

A dPCR assay as described above can be a high-plex dPCR assay. A high-plex dPCR assay can be performed with hundreds of target sequences. For example, a high-plex dPCR assay can be performed with 100, 200, 300, 400, 500, 600, 700, 800, 900, or more target sequences. To further increase the plexity as compared to the mid-plex dPCR assay, one can make use of combinatorics. For example, a sample comprising a plurality of nucleic acid sequences can be pooled together into a single tube. DNA polymerase and target specific primers for each target of interest can be added to the tube, and the plurality of nucleic acid sequences can be extended. After the extension, excess primers can be removed.

Subsequently, a universal forward primer comprising a universal sequence, a forward identifier sequence, a target specific forward primer, and a universal reverse primer comprising a universal sequence, a reverse identifier sequence, and target specific reverse primer can be added to the tube for each target sequence of interest. The forward identifier sequence of the universal forward primer and the reverse identifier sequence of the universal reverse primer are different for each target sequence. There can be an excess of the universal forward primers compared to the universal reverse primers. Additionally, a plurality of probes with a fluorescent label can be added to the tube. A probe of the plurality of probes can comprise a fluorescent label on one end of the probe and a fluorescence quenching moiety on the other end of the probe. For example, a probe has a fluorescent label on the 5′ end of the probe and a quenching moiety on the 3′ end of the probe. Alternatively, a probe can have a quenching moiety on the 5′ end of the probe and a fluorescent label on the 3′ end of the probe. The quenching moiety can quench the fluorescence of the fluorescent label when the probe is in single-stranded form (e.g., not hybridized to a complementary nucleotide sequence), but does not quench the fluorescence of the fluorescent label with the probe is hybridized to a complementary nucleotide strand. Examples of a quenching moiety can include black hole quenchers, Dabcyl, Qxl quenchers, Iowa black FQ, Iowa black RQ, and IrDye QC-1. The quenching moiety can quench the fluorescent label of the probe when in close proximity to the fluorophore, such as when in single-stranded form (e.g., not hybridized to a complementary strand). The plurality of probes, in some cases, comprises a plurality of a hybridization probe pair. A hybridization probe pair can comprise a first probe comprising a fluorophore on the 3′ end and a second probe comprising a fluorophore on the 5′ end. A fluorescence can be generated by exciting the 3′ fluorophore on the first probe and then detecting fluorescence from the 5′ fluorophore on the second probe (e.g., via Forster resonance energy transfer (FRET)). A probe can also comprise a modified base or locked nucleic acid (LNA) to enhance the difference in melting temperature between different identifier sequences. Importantly, different labeled probes with a common fluorescent label as described herein can have different melting temperatures. Different melting temperatures for these different labeled probes with a common fluorescent label can be due to probes comprising different probe sequences having differing numbers of mismatches to the corresponding identifier sequences, different probe sequences having differing lengths of complementarity to the corresponding identifier sequences, different probe sequences having differing percentages of nucleotide base content (e.g., differing percentages of GC content) that are complementary to the corresponding identifier sequence, or any combination thereof. For example, a probe labeled with a first fluorescent label can comprise a probe sequence that is completely complementary to a first forward identifier sequence resulting in a first melting temperature, complementary except for one mismatch to a second forward identifier sequence resulting in a second melting temperature, complementary except for two mismatches to a third forward identifier sequence resulting in a third melting temperature, etc.; a probe labeled with a second fluorescent label can comprise a probe sequence that is completely complementary to a first reverse identifier sequence resulting in a fourth melting temperature, complementary except for one mismatch to a second reverse identifier sequence, resulting in a fifth melting temperature complementary except for two mismatches to a third reverse identifier sequence resulting in a sixth melting temperature, etc.; etc. In some embodiments, a probe labeled with a first fluorescent label can comprise a probe sequence that is complementary to a first forward identifier sequence of a first length resulting in a first melting temperature, complementary to a second forward identifier sequence of a second length resulting in a second melting temperature, complementary to a third forward identifier sequence of third length resulting in a third melting temperature, etc.; a probe labeled with a second fluorescent label can comprise a probe sequence that is complementary to a first reverse identifier sequence of a fourth length resulting in a fourth melting temperature, complementary to a second reverse identifier sequence of a fifth length resulting in a fifth melting temperature, complementary to a third reverse identifier sequence of sixth length resulting in a sixth melting temperature, etc.; etc. In some embodiments, a first length is a different length than the other lengths (e.g., a different length than a second length, a third length, etc.), a second length is a different length than the other lengths (e.g., a different length than a first length, a third length, etc.), a third length is different than the other lengths (e.g., a different length than a first length, a second length, etc.), etc. A length can be the same length if the probes are labeled by different fluorophores (e.g., a probe labeled with a first fluorophore and a probe labeled with a second fluorophore). In some embodiments, a probe labeled with a first fluorescent label can comprise a probe sequence that has a first nucleotide base content and is complementary to a first forward identifier sequence resulting in a first melting temperature, has a second nucleotide base content and complementary to a second forward identifier sequence resulting in a second melting temperature, has a third nucleotide base content and is complementary to a third forward identifier sequence resulting in a third melting temperature, etc.; a probe labeled with a second fluorescent label can comprise a probe sequence that has a fourth nucleotide base content and is complementary to a first reverse identifier sequence resulting in a fourth melting temperature, has a fifth nucleotide base content and complementary to a second reverse identifier sequence resulting in a fifth melting temperature, has a sixth nucleotide base content and completely complementary to a third reverse identifier sequence resulting in a sixth melting temperature, etc.; etc. In some embodiments, a first nucleotide base content is a different than the other nucleotide base contents (e.g., a different nucleotide base content than a second nucleotide base content, a third nucleotide base content, etc.), a second nucleotide base content is a different nucleotide base content than the other nucleotide base contents (e.g., a different length than a first nucleotide base content, a third nucleotide base content, etc.), etc. A nucleotide base content can be the same nucleotide base content if the probes are labeled by different fluorophores (e.g., a probe labeled with a first fluorophore and a probe labeled with a second fluorophore). In some embodiments, a first melting temperature is a different than the other melting temperatures (e.g., a different melting temperature than a second melting temperature, a third melting temperature, etc.), a second melting temperature is a different melting temperature than the other melting temperatures (e.g., a different melting temperature than a first melting temperature, a third melting temperature, etc.), etc. A melting temperature can be the same melting temperature if the probes are labeled by different fluorophores (e.g., a probe labeled with a first fluorophore and a probe labeled with a second fluorophore). A first sequence can be complementary to second sequence if the first sequence is at least 1% 5%, 10%, 20%, 30%, 40%, 50%, 60%, 70%, 75%, 80%, 85%, 90%, 95%, 99% or 100%, or any percentage inbetween, complementary to the second sequence. The number of different fluorescently labeled probes can be the same as the number of fluorescent channels for visualization. The sample in the tube then can undergo digital distribution to partitions as described above (e.g., by high-throughput droplet generation, by spreading of a sample of nucleic acid sequences across a microcapillary array plate, by polymerase colony-based distribution, etc.) to distribute nucleic acid sequences into individual partitions wherein an individual partition comprises a single nucleic acid sequence. Next, nucleic acid sequences in individual partitions undergo a symmetric phase PCR amplification to generate double stranded products with the universal forward primer and universal reverse primer. Then, each individual partition can undergo an asymmetric phase of PCR amplification when the universal reverse primer has been completely used to generate a single stranded product. The probes can then hybridize to the forward identifier sequence of the single stranded amplified target sequence or the reverse identifier sequence of the single stranded amplified target sequence accordingly, and individual partitions can then fluoresce with if the probes are hybridized. Absence of fluorescence can indicate that no amplified target sequence is found in that individual partition.

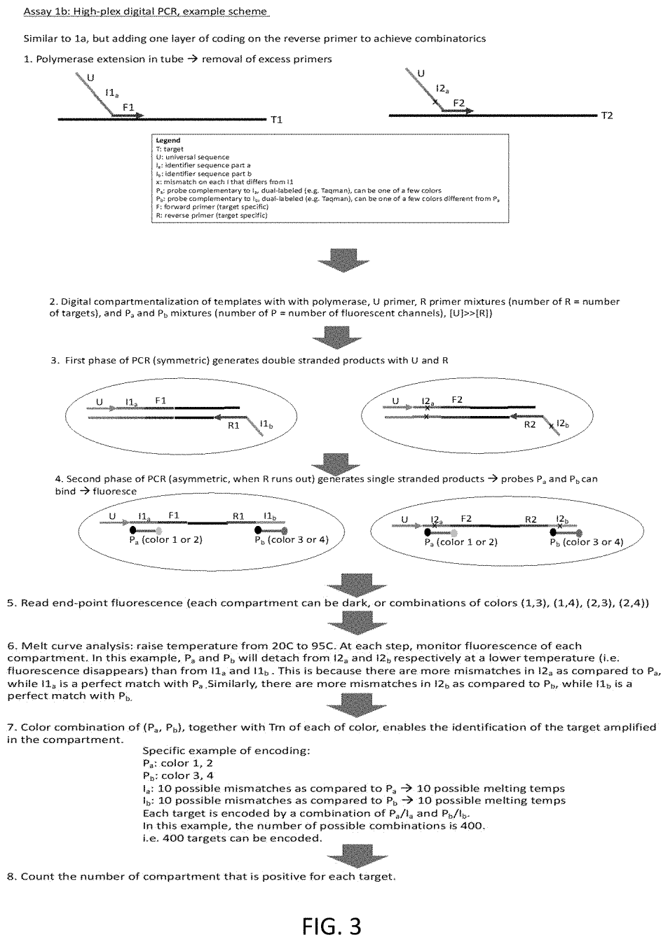

Next, fluorescence can be visualized simultaneously across the plurality of partitions at a plurality of temperatures or during thermal ramping. Thermal ramping can comprise increasing the temperature at intervals of 0.1, 0.2, 0.3, 0.4, 0.5, 0.6, 0.7, 0.8, 0.9, or 1° C. In some examples, the interval is 1, 2, 3, 4, 5, 6, 7, 8, 9, or 10 seconds. For example, a plurality of temperatures can be any temperature from 10° C. to 105° C., 15° C. to 100° C., or 20° C. to 95° C. A plurality of temperatures can be temperatures from thermal ramping, such as increasing a temperature from 10° C. to 105° C., 15° C. to 100° C., or 20° C. to 95° C. At different temperatures, the probes can detach from the identifier sequence of the amplified target sequence with the probes comprising the lowest complementarity detaching at the lowest temperatures, causing the fluorescence to disappear. The probes can detach from the identifier sequence of the amplified target sequence with the next lowest complementarity at a higher temperature, and so on, causing the fluorescence to disappear when the probes detach. The disappearance of fluorescence can be the result of the probe being in single-stranded form, in which the distance between the fluorescent label and the quencher moiety is closer due to the coiling of the probe. Therefore, by combining combination of the color of the fluorescence/loss of fluorescence and the melting temperature of each individual partition, the amplified target sequence in an individual partition can be identified. This can then be followed by counting or quantitating the number of partitions that were positive for a specific amplified target sequence. FIG. 3 illustrates this work flow for 400 targets using four different fluorescent labels for the probes and identifier sequences with 10 possible mismatches to each probes (and thus 10 possible melting temperatures). In some examples, thermal ramping and fluorescence detection occurs in less than 4, 6, 8, 10, 12, 14, 16, 20, 24, 28, 32, 36, 40, 44, or 48 hours.

Additionally, this method can be used for the detection of single nucleotide polymorphisms (SNPs). An additional set of probes that overlap with a SNP can be used to detect the SNP, in which a probe of this additional probe set can bind to an internal region of the amplicon if the SNP is present. The set of probes can have fluorescent labels that are detected by different channels from those used for combinatorial coding described above. Alternatively, this method can be used for the detection of single nucleotide polymorphisms (SNPs) by using a forward primer (F) designed with 3′ end overlapping with a SNP and flanked with a particular identifier sequence, such that only template molecule with the particular SNP is copied and thus detected.

### Short-Read Sequencing Assay Workflow

A sequencing assay as described above can be a short-read sequencing assay. A short-read sequencing assay can be performed using denatured or fragmented nucleic acid sequences. In some instances, the denatured or fragmented nucleic acid sequences are not known prior to using the short-read sequencing assay described herein. A short-read sequencing assay can be performed using pre-defined barcode sequences. In some instances, the short-read sequencing assay using pre-defined barcode sequence is used with dPCR described above to augment the dPCR plexity. For example, the short-read sequencing assay using pre-defined barcode sequence is used after the dPCR described above, before the dPCR described above, or simultaneously with the dPCR described above. The augmented dPCR plexity can be 900; 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; or more targets. The augmented dPCR plexity can from 900 to 10,000; 900 to 20,000; 900 to 30,000; 900 to 40,000; 900 to 50,000; 900 to 60,000; 900 to 70,000; 900 to 80,000; 900 to 90,000; 900 to 100,000; 1,000 to 10,000; 1,000 to 50,000; 1,000 to 100,000; 10,000 to 20,000; 10,000 to 30,000; 10,000 to 40,000; 10,000 to 50,000; 10,000 to 75,000; 10,000 to 100,000 targets. The sequenced denatured or fragmented nucleic acid sequence or sequenced pre-defined barcode sequence can be up to 20, 30, 40, 50, 60, 70, 80, 90, 100, 200, 300, 400, or 500 nucleotides in length. The sequenced denatured or fragmented nucleic acid sequence or sequenced pre-defined barcode sequence can be from 20 to 400, 20 to 300, 20 to 200, 20 to 200, 20 to 100, 20 to 90, 20 to 80, 20 to 70, 20 to 60, 20 to 50, 20 to 40, 20 to 30, 30 to 50, 30 to 60, 30 to 70, 30 to 80, 30 to 90, or 30 to 100 nucleotides in length. The sequence read lengths of the denatured or fragmented nucleic acid sequences or sequenced pre-defined barcode sequences can be up to 20 nucleotides in length or up to 25 nucleotides in length. The sequence read lengths denatured or fragmented nucleic acid sequences or sequenced pre-defined barcode sequences can be up to 30, 40, 50, 60, 70, 80, 90, or 100 nucleotides in length. The sequence read lengths denatured or fragmented nucleic acid sequences or sequenced pre-defined barcode sequences can be from 20 to 100, 20 to 90, 20 to 80, 20 to 70, 20 to 60, 20 to 50, 20 to 40, 20 to 30, 30 to 50, 30 to 60, 30 to 70, 30 to 80, 30 to 90, or 30 to 100 nucleotides in length. The steps of dPCR of a single template sequence, generation of terminated chains of the single template sequence and amplicons thereof, and melt curve analysis of the terminated chains can all be performed in a single partition without any fluid exchange (e.g., no merging or splitting of partitions) as described herein.

For example, a nucleic acid sample can be sheared into a plurality of nucleic acid fragments. The shearing can be performed mechanically or enzymatically. Enzymatic shearing can be performed by nucleases. The nucleic acid fragments can be from 20 to 50, 20 to 100, 20 to 200, 20 to 300, 20 to 400, 20 to 500, 20 to 600, 20 to 700, 20 to 800, 20 to 900, 20 to 1000, 15 to 50, 15 to 100, 15 to 200, 15 to 300, 15 to 400, 15 to 500, 15 to 600, 15 to 700, 15 to 800, 15 to 900, 15 to 1000, 10 to 50, 10 to 100, 10 to 200, 10 to 300, 10 to 400, 10 to 500, 10 to 600, 10 to 700, 10 to 800, 10 to 900, or 1000 nucleotides in length. The nucleic acid sample comprising a plurality of nucleic acid fragments can be pooled together into a single tube. Forward adaptor sequences and reverse adaptor sequences can be attached to the nucleic acid fragments by ligation, in vitro transposition, or multiplex PCR in a tube. The forward or reverse adaptor may contain pre-defined barcode sequences for target DNA identification or for molecule tagging, or may contain random barcode sequences for molecule tagging. Next, a DNA polymerase that incorporates dNTPs preferentially and can be activated after a short hot start, a DNA polymerase that incorporates both dNTPs and ddNTPs and can be activated after a long hot start, dNTPs, fluorescently labeled ddNTPs wherein each type of ddNTP is labeled with a different fluorophore, a forward primer that is complementary to the forward adaptor sequence, and a reverse primer that is complementary to the reverse adaptor sequence can be added to the tube. The dNTPs can be added in excess over the labeled ddNTPs. The forward primer can be added in excess over the reverse primer. Additionally, the forward primer can comprise a quenching moiety either internally or at the 5′ end of the forward primer, can optionally comprise a cleavable site such as a photocleavable site, and can have a higher melting temperature for its complementary sequence than the melting temperature of the reverse primer for its complementary sequence. Examples of a quenching moiety can include black hole quenchers, Dabcyl, Qxl quenchers, Iowa black FQ, Iowa black RQ, and IrDye QC-1. The quenching moiety can quench the fluorophore of the ddNTP when in close proximity to the fluorophore, such as when in single-stranded form (e.g., not hybridized to a complementary strand).

Subsequently, the nucleic acid fragments in the tube can undergo digital distribution to partitions as described above (e.g., either by high-throughput droplet generation or by spreading of the nucleic acid fragments across a microcapillary array plate) to distribute a single nucleic acid fragment into an individual partition. Next, nucleic acid fragments in individual partitions can undergo a symmetric phase of multiple PCR amplifications after a short hot start to generate double stranded products with dNTPs due to activation of the DNA polymerase that preferentially incorporates dNTPs. The reverse primer can be completely used up during the multiple PCR amplifications. Then, individual partitions can undergo an asymmetric phase of PCR amplification after long hot start activate the DNA polymerase that incorporates both dNTPs and ddNTPs to generate a single stranded product. The ddNTPs can also be modified such that they are activated after a long hot start (for instance, through binding to an aptamer or protein). Additionally, the annealing and extension temperature can be raised to reduce the priming of any residual reverse primers. The resulting product can be of various lengths terminated by ddNTPs within each individual partition. The fluorescence of the chain terminated products can be further enhanced and differentiated from background fluorescence of unincorporated ddNTPs via the incorporation of a DNA intercalating dye in the reaction mix. The DNA intercalating dye can be SYBRgreen, EvaGreen, or LCGreen. The DNA intercalating dye can fluoresce when incorporated in double stranded products and act as donor molecules. The emission spectrum of the DNA intercalating dye can overlap with the excitation spectra of the fluorescent moiety of the ddNTPs, thereby increasing the fluorescent intensity of the incorporated ddNTPs via FRET. The forward primer sequence can then be cleaved or partially cleaved from the terminated chains at the cleavage site by a cleavage agent, such as by ultraviolet light. This cleavage of the forward primer can shorten the nucleic acid fragment length, thus increasing the potential differences in melting temperatures for the nucleic acid sequences of various lengths. Often, the cleavage site is 5′ of the quenching moiety so that when the forward primer is partially cleaved, the quenching moiety remains hybridized to the unlabeled strand.

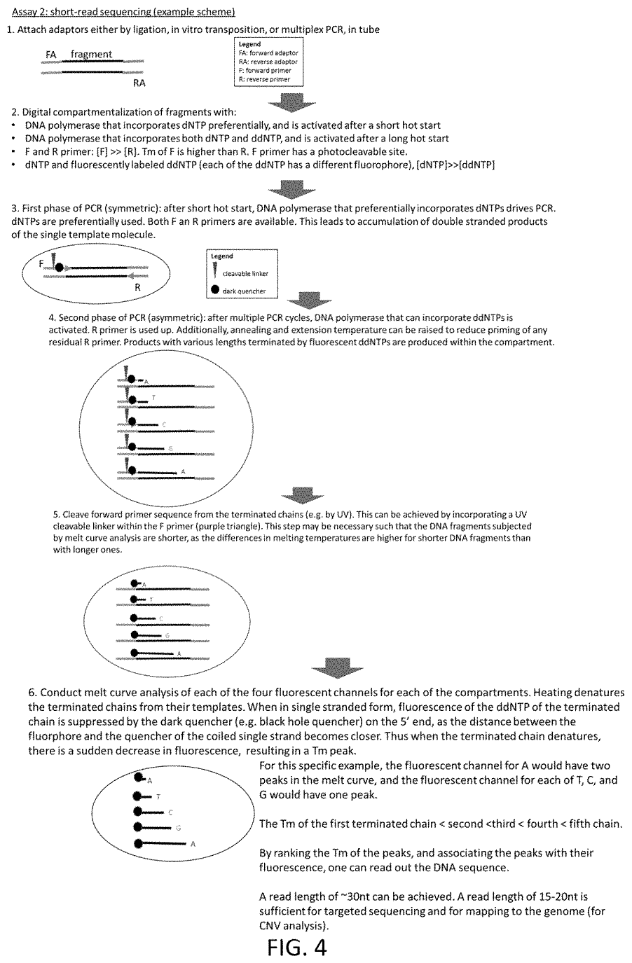

Next, fluorescence can be visualized simultaneously across a plurality of partitions at a plurality of temperatures. Thermal ramping can comprise increasing the temperature at intervals of 0.1, 0.2, 0.3, 0.4, 0.5, 0.6, 0.7, 0.8, 0.9, or 1° C. In some examples, the interval is 1, 2, 3, 4, 5, 6, 7, 8, 9, or 10 seconds. For example, a plurality of temperatures can be any temperature from 10° C. to 105° C., 15° C. to 100° C., or 20° C. to 95° C. A plurality of temperatures can be temperatures from thermal ramping, such as increasing a temperature from 10° C. to 105° C., 15° C. to 100° C., or 20° C. to 95° C. Four fluorescent channels each associated with a nucleotide base and the melting temperature of each nucleic acid sequence of various lengths can be assessed. Since forward primer incorporates a quenching moiety that suppresses fluorescence of ddNTP, when the terminated chain denatures from the unlabeled strand there is a decrease in fluorescence resulting in a melting temperature peak. The shorter nucleic acid sequences can have a lower melting temperature than a longer nucleic acid sequence. For example, the melting temperature of the first terminated chain can be less than melting temperature of the second terminated chain, which can be less than the melting temperature of the third terminated chain, and so on until the end full length nucleic acid fragment, which can have the highest melting temperature. Thus, by detecting and ranking the melting temperature peaks and associating each peak with its fluorescence (indicating the identity of the terminating nucleotide base), one can read out the nucleic acid fragment sequence. A read length of about 15 to 20 nucleotides in a nucleic acid fragment can be allow for mapping of the identified nucleic acid fragment sequence for mapping to the genome, such as for copy number variation (CNV) analysis, aneuploidy detection, or use with RNA-seq studies. FIG. 4 illustrates this work flow for a short-read sequence comprising a nucleic acid fragment. In some examples, thermal ramping and fluorescence detection occurs in less than 4, 6, 8, 10, 12, 14, 16, 20, 24, 28, 32, 36, 40, 44, or 48 hours.

### Fluorophore

A fluorescent label used in mid-plex dPCR assays, in high-plex dPCR assays, or short-read sequencing assays can comprise a fluorophore. A fluorophore can be used in short-read sequencing assays. A fluorophore can be emit light when not in close proximity to a quencher moiety. A fluorophore can be an Alexa Fluor dye, an ATTO dye (e.g., ATTO 390, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho2, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, FAM, HEX, VIC, JOE, TAMRA, ROX, R6G, R110, Yakima Yellow, TET, Pulsar 650, Chromeo 494, or a quantum dot.

### Quenching Moiety

A probe used in mid-plex dPCR assays, in high-plex dPCR assays, or short-read sequencing assays can comprise a quenching moiety. A quenching moiety can be a moiety that quenches the fluorescence of a fluorophore when in close proximity to the fluorophore (e.g., by absorbing energy in the emission spectra of the fluorescent label or fluorophore). Examples of a quenching moiety can include a dark quencher, a Black Hole Quencher (BHQ) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster. For mid-plex dPCR assays and high-plex dPCR assays, the quenching moiety can be at either the 3′ end of the probe or the 5′ end of the probe. For short-read sequencing assays, for example, the quenching moiety is either at the 5′ end of the forward primer or at an internal position in the forward primer. In some examples of short-read sequencing assays, the quenching moiety is at the 3′ to a cleavage site, such as a photo cleavage site.

### Diseases and Conditions

The methods described herein can be useful for the identification of DNA mutations associated with a cancer or tumor. The cancer can comprise breast, heart, lung, small intestine, colon, spleen, kidney, bladder, head, neck, ovarian, prostate, brain, pancreatic, skin, bone, bone marrow, blood, thymus, uterine, testicular and liver tumors. The tumors can comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. The tumor/cancer can be selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, multiple myeloma, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, vipoma, and Wilm's tumor.

The methods described herein can be useful for the identification of DNA mutations associated with a disease or disorder. A disease or disorder can be angelman syndrome, canavan disease, cri du chat, cystic fibrosis, duchenne muscular dystrophy, haemochromatosis, haemophilia, neurofibromatosis, phenylketonuria, prader-willi syndrome, sickle-cell disease, 1p36 deletion syndrome, 18p deletion syndrome, 21-hydroxylase deficiency, Alpha 1-antitrypsin deficiency, AAA syndrome (achalasia-addisonianism-alacrima), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Albinism, Alexander disease, alkaptonuria, Alport syndrome, Alternating hemiplegia of childhood, Amyotrophic lateral sclerosis, Alström syndrome, Alzheimer's disease, Amelogenesis imperfecta, Aminolevulinic acid dehydratase deficiency porphyria, Androgen insensitivity syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dubé syndrome, Brody myopathy, Brunner syndrome, CADASIL syndrome, CARASIL syndrome, Chronic granulomatous disorder, Campomelic dysplasiaX, Carpenter Syndrome, Cerebral dysgenesis-neuropathy-ichthyosis-keratoderma syndrome, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy, types II and XI, Congenital insensitivity to pain with anhidrosis, Cowden syndrome, CPO deficiency (coproporphyria), Cranio-lenticulo-sutural dysplasia, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Di George's syndrome, Distal hereditary motor neuropathies, multiple types, Edwards Syndrome, Ehlers-Danlos syndrome, Emery-Dreifuss syndrome, Erythropoietic protoporphyria, Fanconi anemia (FA), Fabry disease, factor V Leiden thrombophilia, familial adenomatous polyposis, familial dysautonomia, Feingold syndrome, FG syndrome, Friedreich's ataxia, G6PD deficiency, Galactosemia, Gaucher disease, Gillespie syndrome, Glutaric aciduria, type I and type 2, GRACILE syndrome, Griscelli syndrome, Hailey-Hailey disease, Harlequin type ichthyosis, hereditary Hemochromatosis, Hepatoerythropoietic porphyria, Hereditary coproporphyria, Hereditary hemorrhagic telangiectasia (Osler-Weber-Rendu syndrome), Hereditary Inclusion Body Myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, hyperoxaluria, hyperphenylalaninemia, Hypoalphalipoproteinemia (Tangier disease), Hypochondrogenesis, Hypochondroplasia, Immunodeficiency, centromere instability and facial anomalies syndrome (ICF syndrome), Incontinentia pigmenti, Ischiopatellar dysplasia, Isodicentric, Jackson-Weiss syndrome, Joubert syndrome, Juvenile Primary Lateral Sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Lynch Syndrome, lipoprotein lipase deficiency, Maple syrup urine disease, Marfan syndrome, Maroteaux-Lamy syndrome, McCune-Albright syndrome, McLeod syndrome, MEDNIK syndrome, Familial Mediterranean fever, Menkes disease, Methemoglobinemia, methylmalonic academia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Becker type Muscular dystrophy, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, osteogenesis imperfecta, Pantothenate kinase-associated neurodegeneration, Patau Syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic academia, Pitt-Hopkins syndrome, Polycystic kidney disease, Polycystic Ovarian Syndrome (PCOS), Porphyria, Primary ciliary dyskinesia (PCD), primary pulmonary hypertension, protein C deficiency, protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, spondyloepiphyseal dysplasia congenita (SED), Shprintzen-Goldberg syndrome, sickle cell anemia, Siderius X-linked mental retardation syndrome, Sideroblastic anemia, Sly syndrome, Smith-Lemli-Opitz syndrome, Smith Magenis Syndrome, Spinal muscular atrophySpinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous Sclerosis Complex (TSC), Turner syndrome, Usher syndrome, Variegate porphyria, von Hippel-Lindau disease, Waardenburg syndrome, Weissenbacher-Zweymüller syndrome, Williams Syndrome, Wilson disease, Woodhouse-Sakati syndrome, Wolf-Hirschhorn syndrome, Xeroderma pigmentosum, X-linked mental retardation and macroorchidism (fragile X syndrome), X-linked spinal-bulbar muscle atrophy (spinal and bulbar muscular atrophy), Xp11.22 deletion, X-linked severe combined immunodeficiency (X-SCID), X-linked sideroblastic anemia (XLSA), or Zellweger syndrome.

The methods described herein can be useful for the identification of aneuploidy. Aneuploidy can be autosomal aneuploidy or non-autosomal aneuploidy. Autosomal aneuploidy can be for chromosome 13, 18, or 21. Non-autosomal aneuploidy can be for XXX (triple X syndrome), XXXX syndrome (48, XXXX), XXXXX syndrome (49, XXXXX), or XYY syndrome.

### Digital Processing Device

The methods described herein can also include a digital processing device, or use of the same. The digital processing device can include one or more hardware central processing units (CPU) that carry out the device's functions. The digital processing device can further comprise an operating system configured to perform executable instructions. In some instances, the digital processing device is connected to a computer network, is connected to the Internet such that it accesses the World Wide Web, or is connected to a cloud computing infrastructure. In other instances, the digital processing device is connected to an intranet. The digital processing device can be connected to a data storage device.

In accordance with the description herein, suitable digital processing devices can include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers can include those with booklet, slate, and convertible configurations, known to those of skill in the art.

The digital processing device can include an operating system configured to perform executable instructions. The operating system can be, for example, software, including programs and data, which can manage the device's hardware and provides services for execution of applications. Those of skill in the art will recognize that suitable server operating systems can include, by way of non-limiting examples, FreeBSD, Open SD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some cases, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® P53®, Sony® PS4®, Microsoft® Xbox 360®, Microsoft Xbox One, Nintendo® Wii®, Nintendo® Wii U®, and Ouya®.

The device can include a storage and/or memory device. The storage and/or memory device can be one or more physical apparatuses used to store data or programs on a temporary or permanent basis. In some instances, the device is volatile memory and requires power to maintain stored information. The device is non-volatile memory and retains stored information when the digital processing device is not powered. In still other instances, the non-volatile memory comprises flash memory. The non-volatile memory can comprise dynamic random-access memory (DRAM). The non-volatile memory can comprise ferroelectric random access memory (FRAM). The non-volatile memory can comprise phase-change random access memory (PRAM). The device can be a storage device including, by way of non-limiting examples, CD-ROMs, DVDs, flash memory devices, magnetic disk drives, magnetic tapes drives, optical disk drives, and cloud computing based storage. The storage and/or memory device can also be a combination of devices such as those disclosed herein.

The digital processing device can include a display to send visual information to a user. The display can be a cathode ray tube (CRT). The display can be a liquid crystal display (LCD). Alternatively, the display can be a thin film transistor liquid crystal display (TFT-LCD). The display can further be an organic light emitting diode (OLED) display. In various cases, on OLED display is a passive-matrix OLED (PMOLED) or active-matrix OLED (AMOLED) display. The display can be a plasma display. The display can be a video projector. The display can be a combination of devices such as those disclosed herein.

The digital processing device can also include an input device to receive information from a user. For example, the input device can be a keyboard. The input device can be a pointing device including, by way of non-limiting examples, a mouse, trackball, track pad, joystick, game controller, or stylus. The input device can be a touch screen or a multi-touch screen. The input device can be a microphone to capture voice or other sound input. The input device can be a video camera or other sensor to capture motion or visual input. Alternatively, the input device can be a Kinect™, Leap Motion™, or the like. In further aspects, the input device can be a combination of devices such as those disclosed herein.

### Non-Transitory Computer Readable Storage Medium

The methods disclosed herein can include one or more non-transitory computer readable storage media encoded with a program including instructions executable by the operating system of an optionally networked digital processing device A computer readable storage medium can be a tangible component of a digital processing device. A computer readable storage medium can be removable from a digital processing device. A computer readable storage medium can include, by way of non-limiting examples, CD-ROMs, DVDs, flash memory devices, solid state memory, magnetic disk drives, magnetic tape drives, optical disk drives, cloud computing systems and services, and the like. The program and instructions can be permanently, substantially permanently, semi-permanently, or non-transitorily encoded on the media.

### Computer Program

The methods disclosed herein can include at least one computer program, or use of the same. A computer program includes a sequence of instructions, executable in the digital processing device's CPU, written to perform a specified task. Computer readable instructions can be implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

The functionality of the computer readable instructions can be combined or distributed as desired in various environments. A computer program can comprise one sequence of instructions. A computer program can comprise a plurality of sequences of instructions. A computer program can be provided from one location. A computer program can be provided from a plurality of locations. A computer program can include one or more software modules. Sometimes, a computer program can include, in part or in whole, one or more web applications, one or more mobile applications, one or more standalone applications, one or more web browser plug-ins, extensions, add-ins, or add-ons, or combinations thereof.

Computer-implemented systems can be used for the assembly of melting temperature and fluorescence data. An exemplary computer implemented system for assembly comprises a processor, wherein the processor is configured to execute the methods described herein. In an exemplary system, a processor is configured to receive a set of temperature data, receive a set of fluorescence data, assign fluorescence data to a temperature, identify the number of partitions with the same temperature and fluorescence data, and identify the target sequences in the partitions based on the temperature and fluorescence data. In another exemplary system, a processor is configured to receive a set of temperature data, receive a set of fluorescence data, assign fluorescence data to a temperature, identify the base fluorescence and temperature data relative to other base fluorescence and temperature data to determine a nucleic acid sequence, and map the nucleic acid sequence against a reference genome.

### Web Application

A computer program can include a web application. In light of the disclosure provided herein, those of skill in the art will recognize that a web application, in various aspects, utilizes one or more software frameworks and one or more database systems. A web application can be created upon a software framework such as Microsoft® .NET or Ruby on Rails (RoR). A web application can utilize one or more database systems including, by way of non-limiting examples, relational, non-relational, object oriented, associative, and XML database systems. Sometimes, suitable relational database systems can include, by way of non-limiting examples, Microsoft® SQL Server, mySQL™, and Oracle®. Those of skill in the art will also recognize that a web application, in various instances, is written in one or more versions of one or more languages. A web application can be written in one or more markup languages, presentation definition languages, client-side scripting languages, server-side coding languages, database query languages, or combinations thereof. A web application can be written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CS S). A web application can be written to some extent in a client-side scripting language such as Asynchronous Javascript and XML (AJAX), Flash® Actionscript, Javascript, or Silverlight®. A web application can be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. Sometimes, a web application can be written to some extent in a database query language such as Structured Query Language (SQL). Other times, a web application can integrate enterprise server products such as IBM® Lotus Domino®. A web application can include a media player element. A media player element can utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

A computer program can include a mobile application provided to a mobile digital processing device. The mobile application can be provided to a mobile digital processing device at the time it is manufactured. In other cases, the mobile application is provided to a mobile digital processing device via the computer network described herein.

In view of the disclosure provided herein, a mobile application can be created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program can include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. A computer program can include one or more executable complied applications.

### Web Browser Plug-in

The computer program can include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) can be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The methods disclosed herein can include software, server, and/or database modules, or use of the same. In view of the disclosure provided herein, software modules can be created by techniques known to those of skill in the art using machines, software, and languages known to the art. The software modules disclosed herein can be implemented in a multitude of ways. A software module can comprise a file, a section of code, a programming object, a programming structure, or combinations thereof. A software module can comprise a plurality of files, a plurality of sections of code, a plurality of programming objects, a plurality of programming structures, or combinations thereof. The one or more software modules can comprise, by way of non-limiting examples, a web application, a mobile application, and a standalone application. Software modules can be in one computer program or application. Software modules can be in more than one computer program or application. Software modules can be hosted on one machine. Software modules can be hosted on more than one machine. Software modules can be hosted on cloud computing platforms. Software modules can be hosted on one or more machines in one location. Software modules are hosted on one or more machines in more than one location.

### Databases

The methods disclosed herein can include one or more databases, or use of the same. In view of the disclosure provided herein, those of skill in the art will recognize that many databases are suitable for storage and retrieval of analytical information described elsewhere herein. Suitable databases can include, by way of non-limiting examples, relational databases, non-relational databases, object oriented databases, object databases, entity-relationship model databases, associative databases, and XML databases. A database can be internet-based. A database can be web-based. A database can be cloud computing-based. Alternatively, a database can be based on one or more local computer storage devices.

### Services

Methods described herein can further be performed as a service. For example, a service provider can obtain a sample that a customer wishes to analyze. The service provider can then encode the sample to be analyzed by any of the methods described herein, performs the analysis and provides a report to the customer. The customer can also perform the analysis and provide the results to the service provider for decoding. The service provider can then provide the decoded results to the customer. The customer can received encoded analysis of the samples from the provider and can decode the results by interacting with software installed locally (at the customer's location) or remotely (e.g., on a server reachable through a network). The software can generate a report and transmit the report to the costumer. Exemplary customers include clinical laboratories, hospitals, industrial manufacturers, and the like. Sometimes, a customer or party can be any suitable customer or party with a need or desire to use the methods provided herein.

### Server

The methods provided herein can be processed on a server or a computer server. The server can include a central processing unit (CPU, also “processor”) which can be a single core processor, a multi core processor, or plurality of processors for parallel processing. A processor used as part of a control assembly can be a microprocessor. The server can also include memory (e.g., random access memory, read-only memory, flash memory); electronic storage unit (e.g., hard disk); communications interface (e.g., network adaptor) for communicating with one or more other systems; and peripheral devices which includes cache, other memory, data storage, and/or electronic display adaptors. The memory, storage unit, interface, and peripheral devices can be in communication with the processor through a communications bus (solid lines), such as a motherboard. The storage unit can be a data storage unit for storing data. The server can be operatively coupled to a computer network (“network”) with the aid of the communications interface. A processor with the aid of additional hardware can also be operatively coupled to a network. The network can be the Internet, an intranet and/or an extranet, an intranet and/or extranet that is in communication with the Internet, a telecommunication or data network. The network with the aid of the server, can implement a peer-to-peer network, which can enable devices coupled to the server to behave as a client or a server. The server can be capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network. Moreover, a network can be used, for example, to transmit or receive data across an international border.

The server can be in communication with one or more output devices such as a display or printer, and/or with one or more input devices such as, for example, a keyboard, mouse, or joystick. The display can be a touch screen display, in which case it functions as both a display device and an input device. Different and/or additional input devices can be present such an enunciator, a speaker, or a microphone. The server can use any one of a variety of operating systems, such as for example, any one of several versions of Windows®, or of MacOS®, or of Unix®, or of Linux®.

The storage unit can store files or data associated with the operation of a device, systems or methods described herein.

The server can communicate with one or more remote computer systems through the network. The one or more remote computer systems can include, for example, personal computers, laptops, tablets, telephones, Smart phones, or personal digital assistants.

A control assembly can include a single server. The system can include multiple servers in communication with one another through an intranet, extranet and/or the Internet.

The server can be adapted to store device operation parameters, protocols, methods described herein, and other information of potential relevance. Such information can be stored on the storage unit or the server and such data is transmitted through a network.

### EXAMPLES

These examples are provided for illustrative purposes only and not to limit the scope of the claims provided herein.

### Example 1

### Targeted Panels for Cancer DNA Mutations

This example describes the use of dPCR for identifying cancer DNA mutations in a subject. A sample of nucleic acid sequences is provided from a subject. A high-plex dPCR assay is performed for targeted panel of 400 cancer DNA mutations. A plurality of nucleic acid sequences from the sample (e.g., genomic DNA, cDNA, or circulating nucleic acid sequences) are pooled together into a single tube with DNA polymerase and target specific primers for each cancer DNA mutation in the targeted panel. The plurality of nucleic acid sequences are extended as in standard PCR. After the extension, excess primers are removed.

Subsequently, a universal forward primer comprising a universal sequence, a forward identifier sequence, and target specific forward primer, and a universal reverse primer comprising a universal sequence, a reverse identifier sequence, and target specific reverse primer are added to the tube for the cancer DNA mutations of the targeted panel. The forward identifier sequence of the universal forward primer and the reverse identifier sequence of the universal reverse primer are different for each cancer DNA mutation sequence of the targeted panel. An excess of the universal forward primers compared to the universal reverse primers is added to the tube. Additionally, a plurality of probes with a fluorescent label at the 5′ end of the probe and a quenching moiety at the 3′ end of the probe are added to the tubes. More specifically, a probe labeled with a first fluorescent label is comprised of a probe sequence that is completely complementary to a first forward identifier sequence, complementary except for one mismatch to a second forward identifier sequence, complementary except for two mismatches to a third forward identifier sequence, etc.; a probe labeled with a second fluorescent label is comprised of a probe sequence that is completely complementary to a first reverse identifier sequence, complementary except for one mismatch to a second reverse identifier sequence, complementary except for two mismatches to a third reverse identifier sequence, etc.; etc. Digital distribution to partitions by spreading of the sample of nucleic acid sequences across a microcapillary array plate is used to distribute a single nucleic acid sequence into an individual partition across a plurality of partitions. Next, nucleic acid sequences in the individual partitions are amplified by symmetric phase PCR to generate double stranded products with the universal forward primer and universal reverse primer. Then, the individual partitions are amplified by an asymmetric phase of PCR when the universal reverse primer is completely used to generate a single stranded product. The probes are hybridized to the forward identifier sequence of the single stranded amplified target sequence or the reverse identifier sequence of the single stranded amplified target sequence accordingly. A partition is fluorescent if the probes are hybridized. No amplified target sequence is indicated by the absence of fluorescence in that individual partition.

Next, the temperature of each individual partition is raised from 20° C. to 95° C. while simultaneously visualizing fluorescence across the plurality of partitions. The probes with the lowest complementarity to identifier sequence of the amplified target sequence are detached first, causing the fluorescence to disappear due to the close proximity of the fluorescent label and quenching moiety when in single-stranded form. As the temperature increases, the probes with the next lowest complementarity to the identifier sequence of the amplified target sequence are detached and so on, causing the fluorescence to disappear when the probes detach. Therefore, by combining the combination of the color of the fluorescence/loss of fluorescence and the melting temperature of the individual partitions, the amplified target sequences in the individual partitions are identified. This can then be followed by counting the number of partitions that were positive for a specific amplified target sequence. Therefore, the presence or absence of 400 DNA cancer mutations from the targeted panel are identified from the sample from the subject.

### Example 2

### Targeted Panels for Non-Invasive Prenatal Diagnosis of Aneuploidy

This example describes the use of dPCR for identifying non-invasive prenatal diagnosis of aneuploidy in a subject. A sample of nucleic acid sequences (e.g., circulating DNA) is provided from a subject. A mid-plex dPCR assay is performed for targeted panel of fetal aneuploidy for chromosome 13, 18, and 21. A plurality of nucleic acid sequences from the sample are pooled together into a single tube with DNA polymerase and target specific primers for each fetal aneuploidy in the targeted panel. The plurality of nucleic acid sequences are extended as in standard PCR. After the extension, excess primers are removed.

Subsequently, a universal primer comprising a universal sequence, an identifier sequence, and target specific forward primer, and a target specific reverse primer are added to the tube for each fetal aneuploidy in the targeted panel. The identifier sequence of the universal primer is different for each fetal aneuploidy in the targeted panel. An excess of the universal primers compared to the target specific reverse primers are added. Additionally, a plurality of probes with a fluorescent label at the 5′ end of the probe and a quenching moiety at the 3′ end of the probe is added to the tube. A probe labeled with a fluorescent label is comprised of a probe sequence that is completely complementary to an identifier sequence of a first universal primer, complementary except for one mismatch to an identifier sequence of a second universal primer, and complementary except for two mismatches to an identifier sequence of a third universal primer. Digital distribution to partitions by high-throughput droplet generation is used to distribute nucleic acid sequences into individual partitions. Next, nucleic acid sequences in individual partitions are amplified by symmetric phase PCR to generate double stranded products with the universal primer and the target specific reverse primer. Then, individual partitions are amplified by asymmetric phase of PCR when the target specific reverse primer is completely used to generate a single stranded product. The probes are then hybridized to the identifier sequence of the single stranded amplified target sequence, and a partition is fluorescent if the probe is hybridized. No amplified target sequence is indicated by the absence of fluorescence that individual partition.

Next, the temperature of the individual partitions is raised from 20° C. to 95° C. while simultaneously visualizing the fluorescence across the plurality of partitions. The probes with the lowest complementarity to identifier sequence of the amplified target sequence are detached first, causing the fluorescence to disappear due to the close proximity of the fluorescent label and quenching moiety when in single-stranded form. As the temperature increases, the probes with the next lowest complementarity to the identifier sequence of the amplified target sequence are detached and so on, causing the fluorescence to disappear when the probes detach. Therefore, by combining the color of the fluorescence/loss of fluorescence and the melting temperature of the individual partitions, the target amplified sequences in the individual partitions are identified, such as identifying the partitions that comprise a chromosome 13, 18, or 21 nucleic acid sequence. This is then followed by counting the number of partitions that were positive for a specific amplified target sequence, such as a chromosome 13, 18, or 21 nucleic acid sequence. Therefore, the quantitation of chromosome 13, 18, or 21 nucleic acid sequences from the targeted panel are identified from the sample from the subject and are used to diagnosis fetal aneuploidy.

### Example 3

### Whole Genome Shotgun Sequencing for Non-Invasive Prenatal Diagnosis of Aneuploidy

This example describes whole genome shotgun sequencing for non-invasive prenatal diagnosis of aneuploidy. A sample nucleic acids (e.g., circulating DNA) is provided from a subject. The nucleic acids in the sample are then randomly sheared into smaller fragments of about from 20 nucleotides to 400 nucleotides in length. The nucleic acid sample comprising randomly sheared nucleic acid fragments are pooled together into a single tube. Forward adaptor sequences and reverse adaptor sequences are attached to the randomly sheared nucleic acid fragments by ligation, in vitro transposition, or multiplex PCR in a tube. Next, a DNA polymerase that incorporates dNTPs preferentially and is activated after a short hot start, a DNA polymerase that incorporates both dNTPs and ddNTPs and is activated after a long hot start, dNTPs, fluorescently labeled ddNTPs wherein each ddNTP is labeled with a different fluorophore, a forward primer that is complementary to the forward adaptor sequence and a reverse primer that is complementary to the reverse adaptor sequence are added to the tube. The dNTPs are added in excess over the labeled ddNTPs. The forward primer is added in excess over the reverse primer. Additionally, the forward primer is comprised of a photocleavable site and a quenching moiety 3′ of the photocleavable site, and the melting temperature of the forward primer for its complementary sequence is higher than the melting temperature for the reverse primer for its complementary sequence.

Subsequently, the randomly sheared nucleic acid fragments in the tube are distributed by digital distribution into a plurality of partitions using high-throughput droplet generation. Next, the randomly sheared nucleic acid fragments in individual partitions are amplified by a symmetric phase of multiple PCR amplifications after a short hot start to generate double stranded products with dNTPs due to activation of the DNA polymerase that preferentially incorporates dNTPs. The reverse primer is completely used up during the multiple PCR amplifications. Then, individual partitions are amplified by an asymmetric phase of PCR amplification after long hot start activate the DNA polymerase that incorporates both dNTPs and ddNTPs to generate a single stranded product. Additionally, the annealing and extension temperature are raised to reduce the priming of any residual reverse primers. The single stranded products are various lengths terminated by fluorescent ddNTPs within individual partitions. The forward primer sequence is then cleaved from the terminated chains by ultraviolet light. The amplified randomly sheared nucleic acid fragments are shortened by this cleavage of the forward primer, thus the potential differences in melting temperatures for the randomly sheared nucleic acid fragments of various lengths is increased.

Next, the temperature of across the plurality of partitions is raised from 20° C. to 95° C. while simultaneously visualizing the fluorescence in the plurality of partitions for four fluorescent channels. Fluorescence of the terminated chains in suppressed by proximity to the quenching moiety as the terminated chains denature from their complementary strand. A fluorescent channel is associated with a nucleotide base and the melting temperature of nucleic acid sequences of various lengths is assessed. The shorter nucleic acid sequences are expected to have a lower melting temperature than a longer nucleic acid sequence. For example, the melting temperature of the first terminated chain is found to be less than melting temperature of the second terminated chain, which is found to be less than the melting temperature of the third terminated chain, and so on until the end full length randomly sheared nucleic acid fragment, which is found to have the highest melting temperature. Thus, by ranking the melting temperature peaks and associating each peak with its fluorescence (indicating the identity of the terminating nucleotide base), the nucleic acid fragment sequence is identified. A read length of about 15 to 20 nucleotides in a randomly sheared nucleic acid fragments is used to map the identified nucleic acid fragment sequence in the genome. Non-maternal alleles are then identified and the relative proportion of these alleles are assayed across different autosomes. Therefore, the sequence of the nucleic acids from the sample is used to identify fetal aneuploidy.

### Example 4

### RNA-Seq for Gene Expression Analysis of Tissues

This example describes RNA-seq for gene expression analysis of tissues using short-read sequencing. An RNA sample is provided from a subject. A plurality of nucleic acid sequences from the RNA sample are pooled together into a single tube. cDNA is made from RNA via reverse transcription. Forward adaptor sequences and reverse adaptor sequences are attached to the nucleic acid sequences from the cDNA sample by ligation, in vitro transposition, or multiplex PCR in a tube. Next, a DNA polymerase that incorporates dNTPs preferentially and is activated after a short hot start, a DNA polymerase that incorporates both dNTPs and ddNTPs and is activated after a long hot start, dNTPs, fluorescently labeled ddNTPs wherein each ddNTP is labeled with a different fluorophore, a forward primer that is complementary to the forward adaptor sequence and a reverse primer that is complementary to the reverse adaptor sequence are added to the tube. The dNTPs are added in excess over the labeled ddNTPs. The forward primer is added in excess over the reverse primer. Additionally, the forward primer is comprised of a photocleavable site and a quenching moiety 3′ of the photocleavable site, and the melting temperature of the forward primer for its complementary sequence is higher than the melting temperature for the reverse primer for its complementary sequence.

Subsequently, the nucleic acid sequences from the RNA sample and amplicons thereof in the tube are distributed by digital distribution into a plurality of partitions using high-throughput droplet generation, wherein a partition of the plurality comprises a single nucleic acid sequence. Next, nucleic acid sequences from the RNA sample and amplicons thereof in individual partitions are amplified by a symmetric phase of multiple PCR amplifications after a short hot start to generate double stranded products with dNTPs due to activation of the DNA polymerase that preferentially incorporates dNTPs. The reverse primer is completely used up during the multiple PCR amplifications. Then, each individual partition is amplified by an asymmetric phase of PCR amplification after long hot start activate the DNA polymerase that incorporates both dNTPs and ddNTPs to generate a single stranded product. Additionally, the annealing and extension temperature are raised to reduce the priming of any residual reverse primers. The single stranded products are various lengths terminated by fluorescent ddNTPs within each individual partition. The forward primer sequence is then cleaved from the terminated chains by ultraviolet light. The amplified nucleic acid sequences from the RNA sample are shortened by this cleavage of the forward primer, thus the potential differences in melting temperatures for the nucleic acid sequences from the RNA sample of various lengths is increased.

Next, the temperature of across the plurality of partitions is raised from 20° C. to 95° C. while simultaneously visualizing the fluorescence in the plurality of partitions for four fluorescent channels. Fluorescence of the terminated chains in suppressed by proximity to the quenching moiety as the terminated chains denature from their complementary strand. A fluorescent channel is associated with a nucleotide base and the melting temperature of nucleic acid sequences of various lengths is assessed. The shorter nucleic acid sequences are expected to have a lower melting temperature than a longer nucleic acid sequences. For example, the melting temperature of the first terminated chain is found to be less than melting temperature of the second terminated chain, which is found to be less than the melting temperature of the third terminated chain, and so on until the end full length nucleic acid sequences from the RNA sample and amplicons thereof, which is found to have the highest melting temperature. Thus, by ranking the melting temperature peaks and associating each peak with its fluorescence (indicating the identity of the terminating nucleotide base), the nucleic acid sequence sequences from the RNA sample are identified. A read length of about 15 to 20 nucleotides in a nucleic acid sequence from the RNA sample is used to map the identified nucleic acid fragment sequence in the genome. This is then followed by counting the number of partitions that were positive for each amplified RNA sequence. Therefore, the sequence of the RNA from the sample is used to identify and quantitate the gene expression of a tissue from a subject.

### Example 5

### Detection of a Single Nucleotide Polymorphism (SNP)

This example describes the use of dPCR for identifying a single nucleotide polymorphism (SNP) in a subject. A sample of nucleic acid sequences (e.g., circulating DNA) is provided from a subject. A mid-plex dPCR assay is performed for targeted panel of genes including a gene with a specific SNP. A plurality of nucleic acid sequences from the sample are pooled together into a single tube with DNA polymerase and target specific primers for each gene in the targeted panel. The plurality of nucleic acid sequences are extended as in standard PCR. After the extension, excess primers are removed.

Subsequently, a universal primer comprising a universal sequence, an identifier sequence, and target specific forward primer, and a target specific reverse primer are added to the tube for each gene in the targeted panel. The identifier sequence of the universal primer is different for each gene in the targeted panel. For detection of the specific SNP of a gene, a forward primer is comprised of a 3′ end overlapping with the SNP and is flanked by a particular identifier sequence. An excess of the universal primers compared to the target specific reverse primers are added. Additionally, a plurality of probes with a fluorescent label at the 5′ end of the probe and a quenching moiety at the 3′ end of the probe is added to the tube. A probe labeled with a fluorescent label is comprised of a probe sequence that is completely complementary to an identifier sequence of a first universal primer, complementary except for one mismatch to an identifier sequence of a second universal primer, and complementary except for two mismatches to an identifier sequence of a third universal primer. Digital distribution to partitions by high-throughput droplet generation is used to distribute nucleic acid sequences into individual partitions. Next, nucleic acid sequences in individual partitions are amplified by symmetric phase PCR to generate double stranded products with the universal primer and the target specific reverse primer. Then, individual partitions are amplified by asymmetric phase of PCR when the target specific reverse primer is completely used to generate a single stranded product. The probes are then hybridized to the identifier sequence of the single stranded amplified target sequence, and a partition is fluorescent if the probe is hybridized. No amplified target sequence is indicated by the absence of fluorescence that individual partition.

Next, the temperature of the individual partitions is raised from 20° C. to 95° C. while simultaneously visualizing the fluorescence across the plurality of partitions. The probes with the lowest complementarity to identifier sequence of the amplified target sequence are detached first, causing the fluorescence to disappear due to the close proximity of the fluorescent label and quenching moiety when in single-stranded form. As the temperature increases, the probes with the next lowest complementarity to the identifier sequence of the amplified target sequence are detached and so on, causing the fluorescence to disappear when the probes detach. Therefore, by combining the combination of the color of the fluorescence/loss of fluorescence and the melting temperature of the individual partitions, the amplified target sequences in the individual partitions are identified. This can then be followed by counting the number of partitions that were positive for a specific amplified target sequence. For the detection of the specific SNP, detection of the probe that hybridizes to the particular identifier associated with the SNP sequence indicates the presence of the SNP. Therefore, the presence or absence of the genes of the targeted panel are identified from the sample from the subject as well as the presence or absence of a gene with a specific SNP is identified from the sample.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-98. (canceled)

99. A method for nucleic acid sequencing comprising:
a) attaching molecules having a common adaptor to a plurality of nucleic acid fragments to generate a plurality of adaptor-tagged nucleic acid fragments;
b) distributing the plurality of adaptor-tagged nucleic acid fragments into a plurality of partitions, wherein a partition of the plurality of partitions comprises a single adaptor-tagged nucleic acid fragment of the plurality of adaptor-tagged nucleic acid fragments, a primer set, a plurality of nucleotides, and a plurality of labeled, terminated nucleotides;
c) in the plurality of partitions, subjecting the plurality of adaptor-tagged nucleic acids and amplicons thereof to nucleic acid extension reactions with the primer set, plurality of nucleotides, and plurality of labeled, terminated nucleotides; and
d) detecting signals indicative of labeled, terminated nucleotides in the plurality of partitions at a plurality of temperatures,
wherein the signals detected in d) correspond to nucleotide bases and positions of the nucleotide bases in sequences of the nucleic acid fragments.

100. The method of claim 99, wherein the attaching molecules having a common adaptor is by ligation, in vitro transposition, or multiplex polymerase chain reaction (PCR).

101. The method of claim 99, wherein the primer set comprises a forward primer complementary to a region of the molecules having a common adaptor and a reverse primer complementary to a region of the molecules having a common adaptor.

102. The method of claim 101, wherein the forward primer comprises a quenching moiety.

103. The method of claim 102, wherein: (i) the quenching moiety is internal or at the 5′ end of the forward primer or (ii) the quenching moiety is 3′ of the cleavable site.

104. The method of claim 101, wherein the forward primer comprises a cleavable site.

105. The method of claim 99, wherein c) further comprises: (i) amplifying the plurality of adaptor-tagged nucleic acids and amplicons thereof with the primer set and a first DNA polymerase; and (ii) amplifying the plurality of adaptor-tagged nucleic acids and amplicons thereof with the primer set and a second DNA polymerase.

106. The method of claim 105, wherein c) is performed with thermocycling.

107. The method of claim 106, performing annealing and extension at lower temperatures in (i) as compared to (ii).

108. The method of claim 105, wherein (ii) generates terminated nucleic acid sequence amplicons of varying lengths.

109. The method of claim 108, wherein the terminated nucleic acid sequence amplicons of varying lengths have different melting temperatures.

110. The method of claim 99, wherein the plurality of temperatures comprise a temperature from 20° C. to 90° C.

111. The method of claim 99, wherein d) comprises is performed starting at 20° C. and temperature is subsequently increased to 90° C.

112. The method of claim 99, wherein the distributing is droplet-based, array-based, polymerase colony-based, or microfluidic device-based distribution.

113. The method of claim 99, wherein a subset of the signals is detected simultaneously across the plurality of partitions at a temperature of the plurality of temperatures.

114. The method of claim 99, wherein the plurality of nucleic acid fragments are from 10 to 25 nucleotides in length.

115. The method of claim 99, wherein a)-d) occur in a single vessel.

116. The method of claim 99, wherein partitions of the plurality of partitions are arranged in an array.

117. The method of claim 99, wherein partitions of the plurality of partitions are arranged three-dimensionally.

118. The method of claim 99, wherein the detecting signals is non-linear.

119. The method of claim 99, wherein the plurality of partitions comprises at least 10⁵ partitions.

120. The method of claim 99, wherein the partition of the plurality of partitions further comprises a first DNA polymerase and a second DNA polymerase, and wherein c) further comprises i) subjecting the partitions to amplification conditions that activate the first DNA polymerase but not the second DNA polymerase to amplify the plurality of adaptor-tagged nucleic acid sequences and produce amplicons, and ii) subjecting the partitions to amplification conditions that activate the second DNA polymerase to amplify the amplicons.
